(19) United States
(12) Patent Application Publication
(10) Pub. No.: US 2014/0005255 A1
(43) Pub. Date: Jan. 2, 2014

(54) Agonists Of Toll Like Receptor For Treating Cardiovasuclar Disease And Obesity

(71) Applicants: Claudia Monaco, London (GB); Marc Feldmann, London (GB)

(72) Inventors: Claudia Monaco, London (GB); Marc Feldmann, London (GB)

(21) Appl. No.: 13/977,656

(22) PCT Filed: Dec. 29, 2011
(86) PCT No.: PCT/GB2011/052593

§ 371 Date: Sep. 19, 2013

(30) Foreign Application Priority Data
Dec. 29, 2010 (GB) 1022049.9

(51) Int. Cl. A61K 31/7088; A61K 45/06

(52) U.S. Cl. CPC A61K 31/7088; A61K 45/06
USPC 514/44 R; 536/23.1

(57) ABSTRACT

A method for treating or aiding in preventing cardiovascular disease in a patient, comprising the step of administering to the patient a therapeutically effective amount of an agonist of an endosomal TLR. The agonist of the endosomal TLR may be an agonist of TLR3, optionally poly l:poly C12U or Poly (l:C).

The present invention concerns methods, uses and compositions useful in the treatment of patients with or at risk of cardiovascular disease.

The work leading to this invention has received funding from the European Community's Seventh Framework Programme (FP7/2007-2013) under grant agreement no. 201668.

Cardiovascular disease—caused by atherosclerosis and its thrombotic complications—is the world's biggest killer (1). The therapeutic advantage of statins over other lipid lowering strategies (2) supports a complex interaction between risk factors including lipid metabolism, and inflammation. The pathways that sustain disease are beginning to be understood but those that might protect are less clear. These gaps in our knowledge are hampering the generation of new preventative or therapeutic strategies.

In recent years, the interplay between innate immunity—defined as existing in all individuals before exposure to antigen—and adaptive immunity—occurring after antigenic stimulation—have become more appreciated. Charles Janeway's realization that innate immunity triggers adaptive responses (immunology's “dirty” secret) has catalyzed research to analyze innate immune sensors and their effector pathways (3). Since then, a family of at least 13 TLRs has emerged that can recognize the conserved molecular patterns of all microbial classes, bacterial, parasitic and viral, and protect the host from these pathogens. While extracellular TLRs (TLR2, TLR4, TLR5) recognize bacterial wall components, endosomal TLRs recognize nucleic acid patterns belonging to viruses or bacteria including double stranded (ds) RNA (TLR3), single stranded (ss) RNA (TLR7/8) and dsDNA with hypomethylated CpG motifs (TLR9) (4).

Human TLR3 mRNA and amino acid sequences are listed in NCBI accession number NM 003265, and they are also shown in SEQ ID NOs: 1 and 2, respectively, of US 2006/0110746. TLR3 is also described in WO 98/50547.

Not surprisingly, with so few receptors, TLR ligand selectivity is relatively low and so TLRs also recognize self molecules generated during tissue damage and inflammation in the presence or absence of infection. There is increasing evidence that inappropriate activation of TLRs, for instance via endogenous ligands, may contribute to disease. This was first documented for systemic lupus erythematosus (SLE) where TLR9 was implicated by Marshak-Rothstein (5). Subsequent work has documented a role of TLRs and potentially endogenous TLR ligands in inflammatory arthritis, experimental autoimmune encephalomyelitis (EAE) and atherosclerosis as judged by the effects of ablating MyD88 (6-8), the common TLR and interleukin-1 (IL1) signalling adapter, as well as ablating specific TLRs (7-10).

WO 2006/124676, for example, describes methods and compositions for the treatment of autoimmune and inflammatory diseases associated with TLRs and suggests the use of a TLR3 antagonist, rather than agonist, for treating cardiovascular disease.

However, there is also increasing evidence for the antithesis, that TLR signalling might prevent the onset of autoimmune responses. The administration of agonists of TLR3, TLR4, TLR7 and TLR9 prevents spontaneous diabetes in non-obese diabetic mice (11), while TLR4 abrogation increases EAE (12). TLR3, TLR5 and TLR9 signalling exert protection in mouse models of colitis (13-15).

While TLRs were first documented in cells of the innate immune system, there is increasing evidence that structural cells, such as endothelial cells can also acquire TLR expression and respond to their ligands during pathological processes. In atherosclerosis, TLR2 expression has been reported at very early stages of disease on endothelial cells in atherosclerosis-susceptible regions of the aorta (16).

Here we describe the paradoxical findings of endosomal TLR upregulation, for example TLR3 upregulation, in human atherosclerotic tissue-derived smooth muscle cells, leading to the augmented production of several cytokines and chemokines, many of which are pro-inflammatory. Surprisingly in vivo analysis revealed that endosomal TLR, for example TLR3, is involved in protection of the integrity of the vessel wall against intimal and medial injury and the early stages of atherosclerosis. We consider that provision of an endosomal TLR agonist, for example a TLR3 agonist, is useful in treating or preventing cardiovascular disease, for example atherosclerosis, aneurysm or restenosis.

A first aspect of the invention provides an agonist of endosomal Toll Like Receptor (TLR) signalling, for example an agonist of an endosomal Toll Like Receptor (TLR), for treating or aiding in preventing cardiovascular disease.

A second aspect of the invention provides the use of an agonist of endosomal TLR signalling, for example an agonist of an endosomal TLR, in the manufacture of a medicament for treating or aiding in preventing cardiovascular disease.

A third aspect of the invention provides a method for treating or aiding in preventing cardiovascular disease in a patient, comprising the step of administering to the patient a therapeutically effective amount of an agonist of endosomal TLR signalling, for example an agonist of an endosomal TLR.

In an embodiment, administering an agonist of endosomal TLR signalling may also include administering to a patient two or more agonists of endosomal TLR signalling.

In any of these three aspects of the invention the agonist of endosomal TLR signalling may be an agonist of TLR3 signalling. The agonist of an endosomal TLR may be an agonist of TLR3. Alternatively the agonist of endosomal TLR signalling may be an agonist of TLR7, TLR8 or TLR9 signalling. The agonist of an endosomal TLR may be an agonist of TLR7, TLR8 or TLR9.

The agonist may optionally be poly l:poly C12U or Poly (I:C), or other TLR3 agonist indicated below. The agonist may typically be a double-stranded RNA (including a mismatched double stranded RNA) molecule or analogue thereof, examples of which will be well know to those skilled in the art. Poly I:polyC12U and Poly (I:C), for example, are considered to be agonists of TLR3 (and therefore of TLR3 signalling), but it is considered that Poly I:polyC12U and Poly (I:C) may also act as agonists of other endosomal TLRs, for example TLR 7, 8 or 9.

It will be appreciated that an agonist of endosomal TLR signalling (or of a particular endosomal TLR, for example TLR3, signalling) may be a direct agonist of an/the endosomal TLR i.e. may, or may be considered to, interact directly with the TLR (and typically therefore be termed an agonist of that endosomal TLR); or may act on another component of the TLR's signalling pathway, for example a component shown in FIG. 14 or 15. Thus, for example, an agonist of TLR3 signalling may act on TRIF, TRAF3, TBK1 or IKKε. An agonist of TLR7, TLR8 or TLR9 signalling may, for example, act on MyD88, IRAK4, IRAK1 or TRAF6, but this is less preferred since a number of other receptors signal via this pathway

In an embodiment, it may be preferred that the endosomal TLR agonist is a selective TLR agonist, which acts primarily or exclusively on a specific TLR or specific TLR signalling pathway as is well known in the art. Thus, in an embodiment, the TLR3 agonist may be a selective TLR3 agonist, the TLR7 agonist may be a selective TLR7 agonist, the TLR8 agonist may be a selective TLR8 agonist, and the TLR9 agonist may be a selective TLR9 agonist.

In an alternative embodiment, it may be preferred that the endosomal TLR agonist is a non-specific TLR agonist, which acts on more than TLR or TLR signalling pathway as is well known in the art. For example, discussed below are examples of endosomal TLR agonists that are agonists of both TLR3 and TLR9, and agonists of both TLR7 and TLR8.

Many agonists of endosomal TLR signalling, for example endosomal TLR agonists, are known in the art, including those mentioned below. The disclosures of each of the following patents, patent publications and scientific journal articles relating to the manufacture, use and therapeutic applications of endosomal TLR agonists are incorporated herein by reference.

Many suitable TLR3 agonists are known in the art. For example, poly l:poly C12U, also known as Ampligen or rintatolimod or atvogen, is an experimental immunomodulatory double stranded RNA drug developed by Hemispherx Biopharma of Philadelphia, Pa. See, for example Ichinohe et al (2009) Vaccine, 27(45), 6276-6279. Ampligen has been shown to be a selective agonist of TLR3, see US Patent Applications Nos. 2010/0310600 and 2010/0183638.

Ampligen is a preferred member of a class of dsRNA molecules which may be suitable endosomal TLR agonists for the practice of the invention. These include molecules of the general formula rIₙ·r(C₁₁₋₁₄,U)ₙ, rIₙ·(C₁₂,U)ₙ, and rIₙ,r(C₂₉,G)ₙ, in which the value of n is from 4 to 29, as disclosed in U.S. Pat. Nos. 4,024,222; 4,130,641; 5,593,973; 5,683,986; 5,763,417; and 7,678,774 (Hemispherx Biopharma).

Other dsRNA molecules which may be suitable TLR3 agonists for the practice of the invention include the class of ‘rugged’ dsRNA molecules of the general formula ribo(Iₙ).ribo(C₄₋₂₉U)ₙ, ribo(Iₙ).ribo(C₁₁₋₁₄U)ₙ, or ribo(Iₙ).ribo(C₁₂U)ₙ, wherein the strands are comprised of ribonucleotides (ribo) and n is an integer from about 40 to about 40,000 repeats, as disclosed in US Patent Application No. 2010/0160413, especially paragraph

(Hemispherx Biopharma).

WO 2003/090685 describes methods for stimulating TLR3 and TLR4 pathways for inducing anti-microbial, anti-inflammatory and anticancer responses and suggests that a suitable compound may be a TLR ligand selected from a group consisting of bacterial antigen, LPS, lipid A, taxol, viral antigen, RSV F protein (all considered to be TLR4 agonists); and double stranded RNA, imidazoquinoline compounds, and poly l:C (considered to be TLR3 agonists). The latter group may be useful as TLR agonists in embodiments of the invention.

Other agonists of TLR3 that may be useful in embodiments of the invention include Poly-ICR (Poly IC (Polyriboinosinic-polycytidylic acid)-Poly arginine (Nventa Biopharmaceuticals Corporation); high MW synthetic dsRNA IPH31XX compounds, for example IPH3102, which in humans are specific for TLR3 (Innate Pharma S.A; Schering-Plough Corporation); Oragens™, for example Oragen™ 0004, Oragen™ 0033 and Oragen™ 0044 (Temple University); and NS9, a complex of polyinosinic-polycytidylic acid (Nippon Shinyaku Co., Ltd).

The Oragen™ compounds are synthetic analogues of naturally occurring 2′,5′-oligoadenylate analogues, wherein the analogues are typically conjugated to a carrier molecule to enhance cellular uptake (see U.S. Pat. No. 6,362,171).

WO 2009/130616 (Innate Pharma) describes high MW polyAU dsRNA molecules that are TLR3 agonists. WO 2006/054177, WO 2006/054129, WO 2009/130301 and WO 2009/136282 (Institut Gustave Roussy) describe the use of dsRNA TLR3 agonists for treating cancer.

WO 2007/089151 describes stathmin and stathmin-like compounds that are TLR3 agonists. In an embodiment, it may be advantageous to couple a nucleic acid-based agonist to one of these stathmin or stathmin-like agonists.

Moreover, HMGB1 coupled to RNA or DNA is able to facilitate signalling respectively via TLR3, 7 and 8 and TLR9. Thus, in an embodiment, it may be advantageous to couple a nucleic acid-based agonist to HMGB1.

US 2009/0041809 describes locked nucleic acid compositions that are TLR3 agonists, TLR9 agonists, or both TLR3 and TLR9 agonists (Nventa Pharmaceuticals).

A review article concerning TLR3 agonists is Nicodemus & Berek (March 2010) “TLR3 agonists as immunotherapeutic agents”. Immunotherapy. 2(2):137-40.

Many suitable TLR7 and TLR8 agonists are known in the art.

For example, it has been shown that TLR7 and TLR8 recognize viral and synthetic single-stranded RNAs and small molecules, including a number of nucleosides (Diebold, et al. (2004) Science 303: 1529-31). Certain synthetic compounds, the imidazoquinolones, imiquimod (R-837), and resiquimod (R-848) are ligands of TLR7 and TLR8 (Hemmi et al. (2002) Nat. Immunol 3: 196-200; Jurk, et al. (2002) Nat. Immunol 3: 499). In addition, certain guanosine analogues, such as 7-deaza-G, 7-thia-8-oxo-G (TOG), and 7-allyl-8-oxo-G (Ioxoribine), have been shown to activate TLR7 at high concentrations (Lee, et al. (2003) Proc. Natl. Acad. Sci. USA 100:6646-51). However, these small molecules, e.g., imiquimod, are not selective and are known to act through other receptors (Schon, et al. (2006) J. Invest. Dermatol. 126:1338-47).

Certain GU-rich oligoribonucleotides are immunostimulatory and act through TLR7 and TLR8 (Heil et al. (2004) Science 303: 1526-29; WO 03/086280; WO 98/32462) when complexed with N-[1-(2,3-Dioleoyloxy)propyl]-N,N,N trimethylammoniummethylsulfate (DOTAP) or other lipid agents.

Thus, known TLR7 ligands include:

(1) guanosine analogues, such as 7-deazaguanosine and related compounds, including those described in Townsend, (1976) Heterocyclic Chem, 13, 1363, and Seela, et al, (1981) Chem. Ber., 114(10), 3395-3402; 7-allyl, 8-oxo-guanosine (Ioxorabine) and related compounds, including those described in Reitz, et al., (1994) J. Med. Chem., 37, 3561-3578; 7-methyl, 9-deazaguanosine and related compounds including those described in Girgis et al., (1990) J. Med. Chem., 33, 2750-2755; 8-bromoguanosine and other 8-halogen substituted purine compounds including those described in U.S. Pat. No. 4,643,992; 6-amino-9-benzyl-2-butoxy-9H-purin-8-ol, and other 2, 6, 8, 9-substituted purines including those described in Hirota et al., (2002) J. Med. Chem., 45, 5419-5422, Henry et al. (1990) J. Med. Chem. 33, 2127-2130, Michael et al., (1993) J. Med. Chem., 36, 3431-3436, Furneaux et al. (1999) J. Org. Chem., 64(22), 8411-8412; Barrio et al (1996) J. Org. Chem. 61, 6084-6085, U.S. Pat. No. 4,539,205, U.S. Pat. No. 5,011,828, U.S. Pat. No. 5,041,426, U.S. Pat. No. 4,880,784 and WO 94/07904;

(2) imidazoquinolines, including 1-(4-amino-2-ethoxymethyl-imidazo[4,5-c]quinolin-1-yl)-2-methyl-propan-2-ol (imiquimod), as described in WO 94/17043; 1-isobutyl-1H-imidazo[4,5-c]quinolin-4-ylamine (resiquimod) as described in WO 94/17043 and US 2003/0195209, US 2003/0186949, US 2003/0176458, US 2003/0162806, 2003/0100764, US 2003/0065005 and US 2002/0173655); U.S. Pat. No. 5,395,937; WO 98/17279; and

(3) pyrimidine derivatives, including 2-amino-6-bromo-5-phenyl-3H-pyrimidin-4-one (bropirimine), and similar substituted pyrimidines such as those described in Wierenga et al. (1980) J. Med. Chem., 23, 239-240; Fan et al., (1993) J. Heterocyclic Chem., 30, 1273; Skilnick et al. (1986) J. Med. Chem., 29, 1499-1504; Fried et al., (1980) J. Med. Chem., 23, 237-239, and Fujiwara et al. (2000) Bioorg. Med. Chem. Lett. 10(12): 1317-1320.

In addition TLR7 ligands can be readily identified by known screening methods (see, e.g., Hirota et al., (2002) J. Med. Chem., 45, 5419-5422; and Akira et al., (2003) Immunology Letters, 85, 85-95.

US 2008/0171712 describes a novel class of stabilized immune modulatory RNA (SIMRA) compounds which bind to TLR7 and TLR8. SIMRA compounds that specifically activate TLR7, especially the compounds having a structure as set out in Formulas I-IV in Table 2, and specific compounds listed in Table 4, are described in US 2010/0215642 (Idera Pharmaceuticals, Inc).

TLR7 agonists, including lipid-linked TLR7 agonists, are described in US 2010/0210598 (Regents of the University of California, San Diego).

TLR7 agonists, including orally-available-linked TLR7 agonists and TLR7 agonist prodrugs, are described in US 2010/0256169 (Anadys Pharmaceuticals).

Non-selective TLR7 agonists are described in US 2009/0324551 (The Regents of The University of California).

Immunostimulatory polymers that contain sequence-dependent immunostimulatory RNA motifs and methods for their use are described in US 2010/0272785. The sequence-dependent immunostimulatory RNA motifs and the polymers incorporating such motifs are selective inducers of TLR7 and the TLR7-associated cytokine IFN-α (Coley Pharmaceutical).

US 2010/0029585 and WO 2010/014913 (VentiRx Pharmaceuticals) describe formulations of benzo[b]azepine compounds that are TLR7 and/or TLR8 agonists. TLR8 agonists that may be suitable in the context of the present invention include VTX-1463 and VTX-2337 (VentiRx Pharmaceuticals), both of which have successfully completed phase I clinical trials.

A review article concerning TLR8 agonists is Philbin & Levy (2007) “Immunostimulatory activity of Toll-like receptor δ agonists towards human leucocytes: basic mechanisms and translational opportunities”. Biochemical Society Transactions 35(6): 1485-90.

Many suitable TLR9 agonists are known in the art. TLR9 specifically recognises CpG DNA that is unmethylated, and initiates a signalling cascade leading to the production of proinflammatory cytokines. Methylation of the cytosine within the CpG motif strongly reduces the affinity of TLR9. Double stranded (ds) CpG DNA is a weaker stimulator of TLR9 compared to its single stranded (ss) counterpart.

Naturally occurring agonists of TLR9 are described in Smith & Wickstrom (1998) J. Natl. Cancer Inst. 90:1146-1154), and their role in cancer is described in Damiano et al. (2007) Proc. Nat. Acad. Sci. USA 104: 12468-12473.

CPG 7909 is an immunostimulatory TLR9 agonist oligodeoxynucleotide that was found to be well tolerated in a phase I/II clinical study (Cooper et al, (2004) J. Clin. Immunol., 24(6): 693-701).

The CpG enriched, synthetic oligodeoxynucleotide TLR9 agonist PF-3512676 was found to have antilymphoma activity in a phase I/II clinical study (Brody et al (2010) J. Clin. Oncol., 28(28): 4324-32).

Certain TLR9 agonists are comprised of 3′-3′ linked DNA structures containing a core CpR dinucleotide, wherein the R is a modified guanosine (U.S. Pat. No. 7,276,489). In addition, specific chemical modifications have allowed the preparation of specific oligonucleotide analogues that generate distinct modulations of the immune response. In particular, structure activity relationship studies have allowed identification of synthetic motifs and novel DNA-based compounds that generate specific modulations of the immune response and these modulations are distinct from those generated by unmethylated CpG dinucleotides (Kandimalla et al. (2005) Proc. Natl. Acad. Sci. USA 102: 6925-6930; Kandimalla et al. (2003) Proc. Nat. Acad. Sci. USA 100: 14303-14308; Cong et al. (2003) Biochem Biophys Res. Commun. 310: 1133-1139; Kandimalla et al. (2003) Biochem. Biophys. Res. Commun. 306: 948-953; Kandimalla et al. (2003) Nucleic Acids Res. 31: 2393-2400; Yu, D. et al. (2003) Bioorg. Med. Chem. 11:459-464; Bhagat, L. et al. (2003) Biochem. Biophys. Res. Commun. 300:853-861; Yu, D. et al. (2002) Nucleic Acids Res. 30:4460-4469; Yu, D. et al. (2002) J. Med. Chem. 45:4540-4548. Yu, D. et al. (2002) Biochem. Biophys. Res. Commun. 297:83-90; Kandimalla. E. et al. (2002) Bioconjug. Chem. 13:966-974; Yu, D. et al. (2002) Nucleic Acids Res. 30:1613-1619; Yu, D. et al. (2001) Bioorg. Med. Chem. 9: 2803-2808; Yu et al. (2001) Bioorg. Med. Chem. Lett. 11: 2263-2267; Kandimalla et al. (2001) Bioorg. Med. Chem. 9: 807-813; Yu et al. (2000) Bioorg. Med. Chem. Lett. 10: 2585-2588; and Putta et al. (2006) Nucleic Acids Res. 34: 3231-3238).

US 2009/0053206 describes a number of TLR9 agonists, in particular compounds I-169 listed in Table 1; US 2008/0292648 describes a number of TLR9 agonists, in particular compounds I-92 listed in Table 1; and US 2007/0105800 describes oligonucleotide-based compounds that are TLR9 agonists (Idera Pharmaceuticals). Suitable TLR9 agonists may also include the selective TLR9 agonists IMO-2055, IMO-2125 and IMO-2134 that are undergoing phase 1/phase 2 clinical trials (Idera Pharmaceuticals).

US 2010/0016250 describes a number of TLR9 agonists, in particular compounds of Formula I (Kyowa Hakko Kirin Co).

As mentioned above, US 2009/0041809 describes compositions that are TLR9 agonists or both TLR3 and TLR9 agonists (Nventa Pharmaceuticals).

In some embodiments, the endosomal TLR agonist may be a short nucleic acid molecule (whether RNA or DNA or analogues thereof, and whether single or double stranded) of less than 10 or less than 15 or less than 20 or less than 25 or less than 30 or less than 35 or less than 40 or less than 45 or less than 50 (ribo)nucleotides (or pairs when double stranded). Thus in certain embodiments the molecule can be, for example, 10, 11, 12, 13, 14, 15, 16, 17, 18, 19, 20, 21, 22, 23, 24, 25, 26, 27, 28, 29, 30, 31, 32, 33, 34 or 35 (ribo)nucleotides (pairs) long. In an alternative embodiment, the endosomal TLR agonist may be a longer molecule of more than 50, e.g. from 50 to 500 (ribo)nucleotides (pairs), such as from about 50 to about 100 residues, or from about 100 to about 200 residues, or from about 200 to about 300 residues, or from about 300 to about 400 residues, or from about 400 to about 500 residues. In a further embodiment, the endosomal TLR agonist may be a long molecule of 500 to 2500 (ribo)nucleotides (pairs), or more, such as from about 500 to about 1000 residues, or from about 1000 to about 2500 residues, or more.

In an embodiment, an RNA-based agonist may comprise at least one phosphodiester, phosphorothioate or phosphorodithioate interribonucleoside linkage.

In an embodiment, it is preferred that the single or double stranded nucleic acid TLR agonist is not a sequence specific agent, such as an RNAi or antisense RNA or similar, that is designed to interfere with gene expression. Thus, in an embodiment, the endosomal TLR agonist is not a nucleic acid molecule designed to interfere with (i.e., inhibit or prevent) expression of a gene associated with cardiovascular disease.

A commercially available kit containing human TLR3, TLR7, TLR8 and TLR9 agonists can be obtained from InvivoGen. The InvivoGen Human TLR3/7/8/9 Agonist Kit (Code: tlrl-kit3hw2) contains the TLR3 agonists Poly(i:c) and Poly(i:c) LmW; the TLR7 agonists imiquimod and cL264; the TLR7/8 agonists r848 and cL075; the TLR8 agonists ssPolyU/LyoVec™ and ssrna40/LyoVec™; and the TLR9 agonists odn2006, odn2216 and E. coli ssdna/LyoVec™; and suitable controls. Imiquimod (R837), is an imidazoquinoline amine analogue to guanosine, and induces the production of cytokines such as IFN-α. Imiquimod activates only TLR7 but not TLR8. This activation is MyD88-dependent and leads to the induction of the transcription factor NF-kB. CL264 is a novel 9-substituted-8 hydroxyadenine derivative. CL264 induces the activation of NF-kB and the secretion of IFN-α in TLR7-expressing cells. CL264 is a TLR7-specific ligand; it does not stimulate TLR8 even at high concentrations (>10 mg/ml). R848 is an imidazoquinoline compound with potent anti-viral activity. This water soluble, low molecular weight synthetic molecule activates immune cells via the TLR7/TLR8MyD88-dependent signalling pathway, and was shown to trigger NF-kB activation in cells expressing murine TLR8 when combined with poly(dT). CL075 (3M002) is a thiazoloquinolone derivative that stimulates TLR8 in human PBMC. It activates NF-kB and triggers preferentially the production of TNF-α and IL-12. CL075 is about a 10× stronger agonist of TLR8 than for TLR7. ssPolyU/LyoVec™ is a lyophilized preparation of single-stranded poly-uridine (polyU) (i.e., a ssRNA) complexed with the cationic lipid LyoVec™ to protect it from degradation and facilitate its uptake. ssPolyU/LyoVec™. ssRNA40 is a 20-mer phosphothioate protected single-stranded RNA oligonucleotide containing the GU-rich sequence (5′-GCCCGUCUGUUGUGUGACUC-3′; SEQ ID No: 1). ssRNA40 is complexed with the cationic lipid LyoVec™ to protect it from degradation and facilitate its uptake. CpG ODNs are synthetic oligonucleotides containing unmethylated CpG dinucleotides in particular sequence contexts that induce strong immunostimulatory effects through the activation of TLR9. ODN2006 has the sequence: 5′-tcg tcg ttt tgt cgt ttt gtc gtt-3′ (SEQ ID No: 2), and ODN2216 has the sequence: 5′-ggG GGA CGA TCG TCg ggg gg-3′ (SEQ ID No: 3) (bases shown in capital letters are phosphodiester, and those in lower case are phosphorothioate (nuclease resistant). E. coli ssDNA/LyoVec™activates TLR9 similarly to CpG-ODNs in a species-independent manner. It consists of sheared single-stranded DNA fragments produced by treating genomic E. coli DNA with ultrasound followed by heat denaturation. These fragments are complexed with LyoVec™, a lipid-based transfection reagent, to allow penetration of the DNA in the cells.

The skilled person will readily be able to identify a compound as a TLR3 or, as appropriate, TLR7, 8 or 9, agonist by methods well known to those skilled in the art. WO 2003/090685, for example, may describe appropriate methods. Methods described in the present application, for example in Example 1, may also be useful in assessing the ability of a compound to act as a TLR3 agonist. For example, a screening assay for TLR7, TLR8 and TLR9 agonists is described in U.S. Pat. No. 7,498,409 (Schering-Plough Corp).

The patient may be a patient at risk of restenosis; and/or the patient may have or be at risk of atherosclerosis or aneurysm; or other vessel wall damage or loss of vessel wall integrity. The patient typically is not considered to be a patient at such risk merely by having or being at risk of diabetes. Thus, the patient is not a patient selected solely or mainly on the basis of having or being at risk of diabetes.

Biomarkers that may indicate an increased risk of cardiovascular disease include higher fibrinogen and PAI-1 blood concentration, elevated homocysteine, elevated blood levels of asymmetric dimethylarginine, high inflammation as measured by C-reactive protein, and elevated blood levels of brain natriuretic peptide (BNP). Other risk factors for cardiovascular disease include high blood pressure, high LDL cholesterol, low HDL cholesterol, menopause, lack of physical activity or exercise, obesity and smoking.

In most patients diagnosed with atherosclerotic ischemic heart disease via a positive provocative test (e.g. ECG treadmill test or myocardial perfusion scan) or coronary angiography (e.g. immediately after an acute event), there is indication to undergo revasculation to reopen the arterial lumen. The options for revascularization are either surgical or percutaneous. Percutaneous coronary intervention (PCI) with insertion of a metal device called “stent” is often the preferred method of revascularization in the United States and Europe for ischemic heart disease. A total of 1,000,000 PCI are performed annually in the US and a similar number in Europe also annually. The indications for PCI with stenting are also spreading to other vascular beds, including the recent inception of carotid PCI and stenting. However, bare metal stent are associated with an up to 5% risk of subacute thrombosis and up to 30% risk of reocclusion (restenosis) during the first year of treatment. Drug eluting stents are now utilized that allow the sustained local release of an antiproliferative agent at the site of arterial injury. However, recent reports suggest an incremental risk of 0.5% per year for late stent thrombosis with drug eluting stents due to delayed resurfacing with endothelium in the presence of antiproliferative agents. Hence novel drugs targeting restenosis for local or systemic release are needed.

Thus, the endosomal TLR agonist may be administered to a patient (e.g., a human or a non-human mammal) in order to reduce or prevent or aid in the prevention of restenosis that can occur, for example, after angioplasty, stent placement, vascular surgery, cardiac surgery, or interventional radiology.

Methods for identifying a patient that has an increased risk of developing restenosis are described, for example, in WO 2010/139063, WO 2010/097495, WO 2009/073526, WO 2007/131202 and WO 2007/044278.

Aortic aneurysms are particularly frequent in the abdominal aorta and their incidence increases with age. Men of 65 years of age screened via ultrasonography have a prevalence of aortic abdominal aneurysm of 5%. In addition, methods for identifying a patient that has an increased risk of developing a vascular aneurysm are described, for example, in WO 2009/091581 and WO 2009/046267. The aneurysm is associate with a high risk of rupture once it increases its diameter to 5 cm. Rupture of an aneurysm is a dramatic event linked to death prior to reaching the hospital in 25% of cases and an intraoperative mortality of 50%. There is no current treatment to stop aneurysm formation.

Methods for identifying a patient that has an increased risk of developing atherosclerosis and for the early detection of atherosclerosis are described, for example, in WO 2010/113034, WO 2010/102238, WO 2010/064147, WO 2010/045346, WO 2009/101037, WO 2008/049125, WO 2007/102018, WO 2007/095126, WO 2007/002821 and WO 2006/136791.

The patient may be administered the agonist alongside standard therapy for combating risk factors associated with cardiovascular disease (e.g., lipid lowering drugs, oral and injectable antidiabetic treatments and/or blood pressure lowering drugs) and antithrombotic therapy (such as aspirin, clopidogrel, dipyridamole and ticlopidine).

Lipid lowering drugs include the statins, the fibrates, and other drugs, such as ezetimibe, colesevelam, torcetrapib, avasimibe, implitapide and niacin. Reviews of lipid lowering drugs are given by Pahan (2006) Cell Mol Life Sci. 63(10): 1165-1178, and Nair & Darrow (2009) Endocrinol. Metab. Clin. North Am. 38(1): 185-206.

Commonly prescribed blood pressure lowering drugs include ACE inhibitors (e.g., benazepril, captopril, enalapril, fosinopril, lisinopril, moexipril, perindopril, quinapril, ramipril and trandolapril); angiotensin II receptor blockers (e.g., candesartan, eprosartan, irbesartan, losartan, telmisartan and valsartan); beta blockers (e.g., acebutolol, atenolol, betaxolol, bisoprolol/hydrochlorothiazide, bisoprolol, carteolol, metoprolol, nadolol, propranolol, sotalol and timolol); calcium channel blockers (e.g., amlodipine, bepridil, diltiazem, felodipine, nifedipine, nimodipine, nisoldipine and verapamil); and diuretics (amiloride, bumetanide, chlorothiazide, chlorthalidone, furosemide, hydrochlorothiazide, indapamide and spironolactone) (American Heart Association, 2008).

Commonly prescribed antidiabetic therapies include sulfonylureas (e.g., glyburide, glipizide and chlorpropamide); meglitinides (e.g., repaglinide and nateglinide); biguanides (e.g., metformin); alpha-glucosidase inhibitors (e.g., acarbose, and meglitol); thiazolidinediones (e.g., rosiglitazone and pioglitazone); DPP-4 inhibitors (e.g., sitagliptin and saxagliptin); incretin mimetics (e.g., exenatide); pramlintide (a synthetic form of amylin) and insulin (American Diabetes Association).

These additional therapies will usually be administered to the patient by their standard routes of administration and at standard dosages for the patient, as is well known in the art.

Preferably, the patient is a human individual. However, when the patient is other than a human patient, it may be a non-human mammalian individual, such as a horse, dog, pig, cow, sheep, rat, mouse, guinea pig or primate. It is appreciated that the non-human patient may be an animal model of human cardiovascular disease.

It is appreciated that the endosomal TLR agonists for administration to a patient will normally be formulated as a pharmaceutical composition, i.e. together with a pharmaceutically acceptable carrier, diluent or excipient. By “pharmaceutically acceptable” is included that the formulation is sterile and pyrogen free. Suitable pharmaceutical carriers, diluents and excipients are well known in the art of pharmacy. The carrier(s) must be “acceptable” in the sense of being compatible with the compound and not deleterious to the recipients thereof. Typically, the carriers will be water or saline which will be sterile and pyrogen free; however, other acceptable carriers may be used. Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The endosomal TLR agonists may be introduced into cells in the patient using any suitable method, such as those described herein. In an embodiment, the RNA may be protected from the extracellular environment, for example by being contained within a suitable carrier or vehicle. Liposome-mediated transfer, e.g. the oligofectamine method, may be used.

Since the endosomal TLR agonists are for treatment of cardiovascular disease, they can be administered systemically into the circulation where they will reach their desired site of action. Thus, in an embodiment, the pharmaceutical compositions or formulations for administration to a patient are formulated for parenteral administration, more particularly for intravenous administration. In a preferred embodiment, the pharmaceutical composition is suitable for intravenous administration to a patient, for example by injection.

It is also appreciated that methods of targeting and delivering therapeutic agents directly to specific regions of the body are well known to a person of skill in the art. Thus, for example, the agonists may be delivered directly to the heart for the treatment of heart disease (Melo et al (2004) “Gene and cell-based therapies for heart disease.” FASEB J. 18(6): 648-63).

A preferred route of administration is via a catheter or stent. Both synthetic and naturally occurring stent coatings have shown potential to allow prolonged gene elution with no significant adverse reaction (Sharif et al (2004) “Current status of catheter- and stent-based gene therapy.” Cardiovasc Res. 64(2): 208-16). Thus, in an embodiment, the endosomal TLR agonist or a pharmaceutical composition or medicament containing the endosomal TLR agonist, may be delivered as part of a stent or other device, using techniques well known to those skilled in the art.

The endosomal TLR agonist or a pharmaceutical composition or medicament containing the endosomal TLR agonist, can be administered to the patient at any suitable dose. The actual dose will be determined by a physician based upon factors including the agent chosen and the patient characteristics. Administration can be local or systemic. In some embodiments, it can be administered at a dose of at least about 0.01 ng/kg to about 100 mg/kg of body mass (e.g., about 10 ng/kg to about 50 mg/kg, about 20 ng/kg to about 10 mg/kg, about 0.1 ng/kg to about 20 ng/kg, about 3 ng/kg to about 10 ng/kg, or about 50 ng/kg to about 100 μg/kg) of body mass, although other dosages also may provide beneficial results.

In an embodiment, the endosomal TLR agonist or the pharmaceutical composition or medicament containing the endosomal TLR agonist, can be administered as a continuous intravenous infusion beginning at or about the time of reperfusion (i.e., at the time an occluded artery is opened), and continuing for one to seven days (e.g., one, two, three, four, five, six, or seven days). Such a composition can be administered at a dose of, for example, about 0.1 ng/kg/minute to about 500 ng/kg/minute (e.g., about 0.5 ng/kg/minute, about 1 ng/kg/minute, about 2 ng/kg/minute, about 3 ng/kg/minute, about 5 ng/kg/minute, about 10 ng/kg/minute, about 15 ng/kg/minute, about 20 ng/kg/minute, about 25 ng/kg/minute, about 30 ng/kg/minute, about 50 ng/kg/minute, about 100 ng/kg/minute, or about 300 ng/kg/minute). Additionally or alternatively, it may be administered as one or more individual doses starting after reperfusion. For example, a composition can be administered about one hour, about two hours, about three hours, about four hours, about five hours, about six hours, about seven hours, about eight hours, about nine hours, or about ten hours after reperfusion.

In other embodiments, the endosomal TLR agonist or a pharmaceutical composition or medicament containing the endosomal TLR agonist, can be administered before reperfusion (e.g., about one hour prior to reperfusion), either as one or more individual doses or as a continuous infusion beginning about one hour prior to reperfusion). For example, a composition can be administered beginning about one hour, about 45 minutes, about 30 minutes, or about 15 minutes prior to reperfusion.

In some embodiments, the endosomal TLR agonist or a pharmaceutical composition or medicament containing the endosomal TLR agonist, can be administered via a first route (e.g., intravenously) for a first period of time, and subsequently can be administered via another route (e.g., topically or subcutaneously).

For veterinary use, the endosomal TLR agonist is typically administered as a suitably acceptable formulation in accordance with normal veterinary practice and the veterinary surgeon will determine the dosing regimen and route of administration which will be most appropriate for a particular animal.

A fourth aspect of the invention provides an agonist of an endosomal Toll Like Receptor (TLR) for treating or aiding in preventing obesity. The agonist may be an agonist of TLR3, or of TLR7, 8 or 9, as discussed in relation to the preceding three aspects of the invention. Thus, for example, the agonist is optionally poly l:poly C12U or Poly (I:C).

The fourth aspect of the invention may work synergistically with the preceding aspects of the invention in providing benefit to the patient, in that reduction or prevention of obesity may also provide benefit to a patient with or at risk of cardiovascular disease.

In the fourth aspect of the invention, the patient may be administered the agonist in conjunction with standard therapy for combating risk factors (lipid lowering drugs, oral and injectable antidiabetic treatments and/or blood pressure lowering drugs) in order to counteract the unfavourable effect of multiple risk factors upon the cardiovascular system.

Thus, further aspects of the invention provide lipid lowering drugs, oral and injectable antidiabetic treatments and/or blood pressure lowering drugs and/or antithrombotic therapy, as discussed above, for treating or reducing or aiding in preventing cardiovascular disease or obesity in a patient, wherein the patient is administered an agonist of a TLR. Preferences for the TLR and TLR agonist are as indicated above for the appropriate preceding aspect of the invention.

A further aspect of the invention provides a composition or kit of parts comprising an agonist of an endosomal TLR and a lipid lowering drug, an oral or injectable antidiabetic treatment and/or a blood pressure lowering drug, and/or antithrombotic therapy, as discussed above. In an embodiment, when the agonist of an endosomal TLR and the further treatment agent may be administered to a patient by the same route of administration, they may be formulated in the same composition, typically a pharmaceutical composition. More usually, it is not possible or convenient to administer the agonist of an endosomal TLR and the further treatment agent together by the same route of administration. In such cases, the treatment agents may be formulated separately and provided in a kit of parts for separate administration. It may be also preferred that the composition or kit of parts is provided together with instructions for using the treatment agents contained therein for treating or reducing or aiding in preventing cardiovascular disease or obesity in a patient, as discussed above.

A further aspect of the invention provides a method for selecting a compound expected to be useful in treating or aiding in preventing cardiovascular disease or obesity, the method comprising the step of selecting a compound that is an agonist of an endosomal TLR. Cells isolated from human atherosclerotic plaques can be utilized to screen for compounds and to compare their biological activity relative to the dsRNA Polyl:C. The methods in Example 1 describe how this is achieved.

A still further aspect of the invention provides a method for identifying a compound that is, or that is expected to be, useful in treating or reducing or aiding in preventing cardiovascular disease or obesity, the method comprising the steps of (i) selecting a compound that is an agonist of an endosomal TLR, and (ii) testing the selected compound in a model of cardiovascular disease or obesity. Many endosomal TLR agonists that can be selected in step (i) of this method are known, including those discussed above.

In an embodiment, step (i) may comprise identifying a compound as being an agonist of an endosomal TLR, and the identified compound selected for testing in step (ii). Methods for identifying whether a compound is an agonist of an endosomal TLR are well known in the art. In this embodiment, the compounds selected for testing in step (i) may be of a type that is known to be an agonist of an endosomal TLR, such as dsRNA, ssRNA and CpG DNA.

In these screening aspects of the invention, the agonist of an endosomal TLR may be an agonist of TLR3, 7, 8 and/or 9 as discussed above. In an embodiment, a TLR3 agonist may be preferred. In an embodiment, a selective endosomal TLR agonist may be preferred.

Many suitable in vitro and in vivo models of cardiovascular disease and obesity are known in the art and described herein, and include cellular models and animal models of the human diseases.

In an embodiment, it may be preferred if the test agent used in these screening methods is a small molecule (e.g. with a molecule weight less than 5000 daltons, for example less than 4000, 3000, 2000 or 1000 daltons, or with a molecule weight less than 500 daltons, for example less than 450 daltons, 400 daltons, 350 daltons, 300 daltons, 250 daltons, 200 daltons, 150 daltons, 100 daltons, 50 daltons or 10 daltons).

In many instances, high throughput screening of test agents is preferred and the method may be used as a “library screening” method, a term well known to those skilled in the art. Thus, the test agent may be a library of test agents. Methodologies for preparing and screening such libraries are known in the art.

It is appreciated that in the screening methods described herein, which may be drug screening methods, a term well known to those skilled in the art, the test agent may be a drug-like compound or lead compound for the development of a drug-like compound. The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, molecular biology or biochemistry, and is preferably a small molecule, which may be of less than 5000 daltons and which may be water-soluble. A drug-like compound may additionally exhibit improved selectivity and bioavailability, but it will be appreciated that these features may not be essential. The term “lead compound” is similarly well known to those skilled in the art, and may include the meaning that the compound, whilst not itself suitable for use as a drug (for example because it is only weakly potent against its intended target, non-selective in its action, unstable, poorly soluble, difficult to synthesise or has poor bioavailability) may provide a starting-point for the design of other compounds that may have more desirable characteristics.

In an embodiment of the screening methods, an agent identified as a result of the initial screen may be modified and retested.

In a further embodiment of the screening methods, a compound having or expected to have similar properties to an agent identified as a result of the method may be tested.

In a still further embodiment, an agent that has been successfully tested in a cellular model of cardiovascular disease or obesity is further tested in an animal model.

In a still yet further embodiment of the screening methods, an agent that has been identified as a result of the method, and having successfully completed testing in cellular and/or animal models, is further tested for efficacy and safety in a clinical trial for cardiovascular disease or obesity, optionally together with other suitable treatments for the condition.

In a preferred embodiment, an agent that has been identified as a result of carrying out the screening methods is synthesised and purified. Typically, the synthesis and purification is carried out to pharmaceutically acceptable standards.

In a further preferred embodiment, an agent that has been identified as a result of carrying out the screening methods is packaged and presented for use in medicine, and preferably presented for use in treating of cardiovascular disease or obesity.

Further aspects of the invention provide an agonist of a cytosolic pattern recognition receptor, such as MDA5, RIG-I and LPG2, for treating or aiding in preventing cardiovascular disease; or the use of an agonist of MDA5, RIG-I and/or LPG2 in the manufacture of a medicament for treating or aiding in preventing cardiovascular disease; or a method for treating or aiding in preventing cardiovascular disease in a patient, comprising the step of administering to the patient a therapeutically effective amount of an agonist of MDA5, RIG-I and/or LPG2. Agonists of MDA5, RIG-I and LPG2 are considered to include double stranded nucleic acids, for example double stranded RNA and analogues thereof (Kato et al (2006) Nature 441: 101-105; Yoneyama et al (2004) Nature Immunol. 5: 730-7; Loo et al (2008) J. Virol. 82(1): 335-345; and Sato et al (2010) Proc. Natl. Acad. Sci. USA 107(4): 1512-17).

Still further aspects of the invention provide a double stranded nucleic acid, for example a double stranded RNA or analogue thereof, for treating or aiding in preventing cardiovascular disease; or the use of a double stranded nucleic acid, for example a double stranded RNA or analogue thereof, in the manufacture of a medicament for treating or aiding in preventing cardiovascular disease; or a method for treating or aiding in preventing cardiovascular disease in a patient, comprising the step of administering to the patient a therapeutically effective amount of a double stranded nucleic acid, for example a double stranded RNA or analogue thereof. The double stranded nucleic acid is not a sequence specific agent, such as a small interfering RNA (siRNA) or similar. Thus, the dsRNA molecule is not a molecule designed to interfere with (i.e., inhibit or prevent) expression of a gene associated with cardiovascular disease.

Another aspect of the invention provide a single stranded nucleic acid, for example a single stranded RNA or analogue thereof, for treating or aiding in preventing cardiovascular disease; or the use of a single stranded nucleic acid, for example a single stranded RNA or analogue thereof, in the manufacture of a medicament for treating or aiding in preventing cardiovascular disease; or a method for treating or aiding in preventing cardiovascular disease in a patient, comprising the step of administering to the patient a therapeutically effective amount of a single stranded nucleic acid, for example a single stranded RNA or analogue thereof. The single stranded nucleic acid is not a sequence specific agent, such as an antisense RNA or similar. Thus, the single stranded RNA molecule is not a molecule designed to interfere with (i.e., inhibit or prevent) expression of a gene associated with cardiovascular disease.

Long dsRNA is recognised by TLR3, whereas shorter versions are recognised by MDA5, RIG-I and LPG2. Thus, in some embodiments, the ssRNA or dsRNA molecule may be a short molecule of less than 10 or less than 15 or less than 20 or less than 25 or less than 30 or less than 35 or less than 40 or less than 45 or less than 50 ribonucleotides (or pairs when double stranded). Thus in certain embodiments the molecule can be, for example, 8, 9, 10, 11, 12, 13, 14, 15, 16, 17, 18, 19, 20, 21, 22, 23, 24, 25, 26, 27, 28, 29, 30, 31, 32, 33, 34 or 35 ribonucleotides (pairs) long. In an alternative embodiment, the dsRNA may be a longer molecule of 50 to 500 ribonucleotides (pairs), such as from about 50 to about 100 residues, from about 100 to about 200 residues, or from about 200 to about 300 residues, or from about 300 to about 400 residues, or from about 400 to about 500 residues. In a further embodiment, the dsRNA may be a long molecule of 500 to 2500 ribonucleotides (pairs), or more, such as from about 500 to about 1000 residues, or from about 1000 to about 2500 residues, or more.

In an embodiment, the oligoribonucleotide may comprises at least one phosphodiester, phosphorothioate or phosphorodithioate interribonucleoside linkage.

Any document referred to herein is hereby incorporated by reference.

The listing or discussion of an apparently prior-published document in this specification should not necessarily be taken as an acknowledgement that the document is part of the state of the art or is common general knowledge.

The invention is now described in more detail by reference to the following, non-limiting, Figures and Examples.

### FIGURE LEGENDS

FIG. 1 AthSMC exhibit enhanced expression and response to TLR3 A) Concentration of IL-6 in the supernatants of SMC stimulated with various TLR agonists for 24 hours are shown. Bars show mean±SEM (n=6 donors each group). AthSMC displayed enhanced expression of IL-6 when stimulated with Poly(I:C) and FSL-1 compared to AoSMC (**p<0.01, ***p<0.001 respectively; Rank ANCOVA). B) The same data as in A) are shown here as fold change in IL-6 production between AthSMC and AoSMC following TLR agonist stimulation. Bars show mean±SEM (n=6 donors each group). C) AthSMC and AoSMC were stimulated for 5 hours with 25 μg/ml Poly(I:C) or left unstimulated. Genes induced by dsRNA in AoSMC (n=3) and AthSMC (n=7) were examined by quantitative PCR analysis using an Atherosclerosis RT2 Profiler PCR Array (SA Biosciences). Data are shown as mean±SEM. Genes with a fold regulation>2 are shown here. AthSMC displayed an enhanced expression of the indicated genes when stimulated with Poly(I:C) (*p<0.05, **p<0.01, ***p<0.001; paired T test vs. unstimulated). D) Atherosclerosis-related and TLR-pathway-related genes were assessed using quantitative PCR gene arrays (SA Biosciences) using cDNA from unstimulated AoSMC and AthSMC. Genes with a statistical significant upregulation>2 fold are displayed here. Data shown are mean fold-changes of gene expression±SEM of AthSMC (n=9) vs. AoSMC (n=4) (*p<0.05, **p<0.01, ***p<0.001 AthSMC vs. AoSMC; paired t-test). Abbreviations: BIRC3: Baculoviral IAP repeat-containing 3, CCL2: Chemokine (C—C motif) ligand 2, CCL5: Chemokine (C—C motif) ligand 5, ICAM1: Intercellular adhesion molecule-1, LIF: Leukemia inhibitory factor, SELE: E-Selectin, VCAM1: Vascular cell adhesion molecule-1.

FIG. 2 Aortic gene expression of both pro- and anti-inflammatory factors is induced by Poly(I:C) stimulation. 10- and 30-week old C57BL/6, ApoE−/− and TLR3−/− mice were stimulated with PBS or 250 μg Poly(I:C) in PBS. 24 hours post-stimulation, mice were sacrificed, aortas harvested and RNA extracted. Gene expression of CCL5 (A), IL-10 (B), VCAM-1 (C) and IFNβ (D) was examined by quantitative RT-PCR. Bars show overall mean±SEM (n=3-5 mice per group; *p<0.05, **p<0.01, ***p<0.001 PBS v. Poly(I:C); unpaired student's t-test).

FIG. 3 TLR3 activation protects against neointima formation in response to carotid collar injury. A) Representative photomicrographs of injured carotid arteries from C57BL/6 and TLR3^{-/-} mice treated with PBS or Poly(I:C) stained for elastin and counterstained with hematoxylin. Scale bars 200 μm. B & C) Intima/media ratio (IMR) of carotid arteries 21 days after injury from C57BL/6 (B) and TLR3^{-/-} (C) mice treated with PBS or Poly(I:C). Each dot represents the mean IMR per individual mouse. Line represents the mean IMR per group (n=8-11; *** p<0.001; PBS v. Poly(I:C); unpaired student's t-test).

FIG. 4 TLR3 activation protects against elastic lamina interruptions during carotid collar injury. A) Representative photomicrographs of injured carotid arteries from C57BL/6 and TLR3^{-/-} mice treated with PBS stained for elastin and counterstained with hematoxylin. Arrows denote area of breakage of the elastic laminae. Scale bars 200 μm. B) Table detailing number of mice in which a breakage in the elastic lamina was observed (n=8-10 mice per group; *p<0.05 C57BL/6 PBS v. TLR3^{-/-} PBS; §p<0.05 TLR3^{-/-} PBS v. TLR3^{-/-} Poly(I:C); Chi-square test) and the average size of observed breaks (n=8-10 mice per group; **p<0.01 C57BL/6 PBS v. TLR3^{-/-} PBS; §p<0.05 TLR3^{-/-} PBS v. TLR3^{-/-} Poly(I:C); Mann-Whitney U test). C) Graph showing the average number of segments of the injured carotid artery of the five examined for each mouse that were affected or unaffected by elastic lamina breakage (n=8-10 mice per group; ***p<0.001 C57BL/6 PBS v. TLR3^{-/-} PBS; §§§p<0.001 TLR3^{-/-} PBS v. TLR3^{-/-} Poly(I:C); Chi-square test).

FIG. 5 TLR3 deficiency accelerates early atherosclerotic lesion development in the aortic root. A) Representative photomicrographs of aortic roots from 15-week ApoE^{-/-} and ApoE^{-/-}TLR3^{-/-} mice stained with Oil Red 0 and hematoxylin. Scale bars 500 μm. B) Cross-sectional aortic root lesion area (×10³ μm²) in 15-week ApoE^{-/-} and ApoE^{-/-}TLR3^{-/-} mice. C) Cross-sectional aortic root lesion area (%) in 15-week ApoE^{-/-} and ApoE^{-/-}TLR3^{-/-} mice. B&C) Each dot represents the mean lesional area per individual mouse. Line represents the mean lesional area per group (n=7-8; * p<0.05; unpaired student's t-test).

FIG. 6 (A-D) AthSMC exhibit increased cytokine responses to TLR3 stimulation. A&C) Concentration of IL-8 (A) and CCL2/MCP-1 (C) in the supernatants of SMC stimulated with various TLR agonists as shown for 24 hours. Bars show mean±SEM (n=6 donors each group) AthSMC displayed enhanced expression of IL-8 and CCL2/MCP-1 when stimulated with Poly(I:C) and increased IL8 also when stimulated with FSL-1 when compared to AoSMC (**p<0.01, ***p<0.001; Rank ANCOVA). B&D) The same data as in A&C) are shown here as fold change in IL-8 (B) and CCL2/MCP-1 (D) production between AthSMC and AoSMC following TLR agonist stimulation. Bars show mean±SEM (n=6 donors each group).

(E-1) AthSMC Express Increased Intracellular TLR3 Compared to AoSMC.

E&F) Analysis of intracellular TLR3 expression by flow cytometry showed that TLR3 expression was higher on AthSMC (F) compared to AoSMC (E). A representative staining out of 3 separate experiments is shown here. TLR staining was only present after permeabilization, but not on the cell surface. G&H) TLR3 expression was augmented by stimulation with 10 ng/mL IFNs and 25 μg/mL Poly(I:C). AoSMC (B) or AthSMC(C) were stimulated with the indicated agonists for 5 hours before total cellular RNA was extracted and quantitative PCR performed. Data are shown as mean±SEM (n=3 donors each group; *p<0.05, ***p<0.001 vs. unstimulated; One-way analysis of variance with Dunnett's multiple comparison test). I) Type I interferons are produced in the mixed cell culture population in response to TLR9 stimulation. Carotid endarterectomy specimens were digested with an enzymatic mixture and the cell culture population was placed in culture for 24 hours in the presence or absence of 25 μg/ml Poly(I:C) or 1 μM CpG ODN2006 or 1 μM CpG ODN2006 control. IFNα was detected by ELISA. A representative experiment is shown out of 3 that were performed.

FIG. 7 Induction of pro- and anti-inflammatory factors following Poly(I:C) stimulation in aortas. 10- and 30-week old C57BL/6, ApoE−/− and TLR3−/− mice were stimulated with PBS or 250 μg Poly(I:C) in PBS. 24 hours post-stimulation, mice were sacrificed, aortas harvested and RNA extracted. Gene expression of CCL2 (A) and PDL2 (B) was examined by quantitative RT-PCR. Bars show overall mean±SEM (n=3-5 mice per group; **p<0.01, ***p<0.001 PBS v. Poly(I:C); unpaired student's t-test). Abbreviations: PD-L2: programmed death-ligand 2.

FIG. 8 Carotid gene expression of pro-inflammatory factors is induced by Poly(I:C) stimulation. 10- and 30-week old C57BL/6, ApoE^{-/-} and TLR3^{-/-} mice were stimulated with PBS or 250 μg Poly(I:C). 24 hours post-stimulation, mice were sacrificed, carotid arteries harvested and RNA extracted. Gene expression of CCL5 (A), CCL2 (B) and VCAM1 (C) in the carotid arteries was examined by quantitative RT-PCR. Bars show overall mean±SEM (n=4-5 mice per group; *p<0.05, ***p<0.001 PBS v. Poly(I:C); unpaired student's t-test). Abbreviations: CCL2: Chemokine (C—C motif) ligand 2, CCL5: Chemokine (C—C motif) ligand 5, VCAM1: Vascular cell adhesion molecule-1.

FIG. 9 TLR3 gene expression is induced in murine aortas and carotids following stimulation with Poly(I:C). 10- and 30-week old C57BL/6, ApoE^{-/-} and TLR3^{-/-} mice were stimulated with PBS or 250 μg Poly(I:C). 24 hours post-stimulation, mice were sacrificed, aortas and carotid arteries harvested and RNA extracted. TLR3 gene expression was examined in the aorta (A) and the carotid arteries (B) by quantitative RT-PCR. Bars show overall mean±SEM (n=3-5 mice per group; *p<0.05, **p<0.01, ***p<0.001 PBS v. Poly(I:C); unpaired student's t-test).

FIG. 10 Gene expression of anti-inflammatory factors is increased in aortas and lymphoid tissues in C57BL/6 mice treated with Poly(I:C) Aortas, spleens and para-aortic lymph nodes (PALN) of C57BL/6 mice that underwent carotid artery injury and PBS or Poly(I:C) treatment were collected at sacrifice. RNA was extracted and gene expression of CCL5, IFNβ, PD-L1, PD-L2, and IL-10 in the aorta (A), spleen (B) and PALN(C) was examined by RT-PCR. Bars show overall mean±SEM (n=4 mice per group, *p<0.05, **p<0.01 PBS v. Poly(I:C); unpaired student's t-test). Abbreviations: IFNβ: Interferon beta, IL-10: Interleukin-10, PD-L1: programmed death-ligand 1, PD-L2: programmed death-ligand 2.

FIG. 11 TLR3 deficiency does not affect late atherosclerotic lesion development in the aortic root. A) Representative photomicrographs of aortic roots from 30-week ApoE^{-/-} and ApoE^{-/-}TLR3^{-/-} mice stained with Oil Red 0 and hematoxylin. Scale bars 500 μm. B) Cross-sectional aortic root lesion area (×10³ μm²) in 30-week ApoE^{-/-} (n=6) and ApoE^{-/-}TLR3^{-/-} mice (n=8). C) Cross-sectional aortic root lesion area (%) in 30-week ApoE^{-/-} and ApoE^{-/-}TLR3^{-/-} mice. B&C) Each dot represents the mean lesional area per individual mouse. Line represents the mean lesional area per group (n=6-8; p>0.05; unpaired student's t-test).

FIG. 12 TLR3 deficiency does not affect lesional macrophage content. A) Representative photomicrographs of aortic roots from 15- and 30-week ApoE^{-/-} and ApoE^{-/-}TLR3−/− mice stained with an antibody against CD68 for macrophages and counterstained with hematoxylin. Scale bars 500 μm. B & D) Aortic root lesion area staining positive for CD68 (×10³ μm²) in 15- (B) and 30-week (D) ApoE^{-/-} and ApoE^{-/-} TLR3^{-/-} mice. C & E) Aortic root lesion area staining positive for CD68 (%) in 15- (C) and 30-week (E) ApoE^{-/-} and ApoE^{-/-}TLR3^{-/-} mice. B-E) Each dot represents the mean area staining positive per individual mouse. Line represents the mean area staining positive per group (n=6-8; p>0.05; unpaired student's t-test).

FIG. 13 TLR3 deficiency does not affect lesional collagen content. A) Representative photomicrographs of aortic roots from 15- and 30-week ApoE^{-/-} and ApoE^{-/-}TLR3^{-/-} mice stained with masson trichrome staining for collagen (green) and muscle (pink). Scale bars 500 μm. B & D) Aortic root lesion area staining positive for collagen (×10³ μm²) in 15- (B) and 30-week (D) ApoE^{-/-} and ApoE^{-/-}TLR3^{-/-} mice. C & E) Aortic root lesion area staining positive for collagen (%) in 15- (C) and 30-week (E) ApoE^{-/-} and ApoE^{-/-}TLR3^{-/-} mice. B-E) Each dot represents the mean area staining positive per individual mouse. Line represents the mean area staining positive per group (n=6-8; p>0.05; unpaired student's t-test)

FIG. 14 The IL-1/TLR superfamily. This is a schematic showing the members of the IL-1/TLR superfamily and their signalling pathways in the cell.

FIG. 15 Viral genome sensing. This is a schematic of the molecular apparatus for sensing a viral genome in a cell

FIG. 16 Development of obesity in ApoE−/−TLR3−/− mice. ApoE−/−TLR3−/− mice on a high fat diet were significantly more obese than ApoE−/− mice on a high fat diet.

FIG. 17 Role of TLR-3 in arterial injury. This shows the surgical procedure used for assessing the effect of dsRNA TLR3 agonists on arterial injury.

FIG. 18 Role of TLR-3 in atherosclerosis development. This is a schematic showing the outline of the experimental procedures used.

FIG. 19 No effect on monocyte/macrophage content. ApoE−/−TLR3−/− mice and ApoE−/− mice show no significant differences in monocyte/macrophage content in atherosclerotic lesions measured using CD86 as a marker.

### EXAMPLE 1

### The Unexpected Protective Role of TLR3 in the Arterial Wall

The critical role of Toll-like receptors (TLRs) in mammalian host defense has been extensively explored in recent years. The capacity of about 10 TLRs to recognize conserved patterns on many bacterial and viral pathogens is remarkable. With so few receptors, cross reactivity with self-tissue components often occurs. Previous studies have frequently assigned detrimental roles to TLRs, in particular TLR2 and TLR4, in immune and cardiovascular disease. Using human and murine systems, we have investigated for the first time the consequence of TLR3 signalling in vascular disease. We compared the responses of human atheroma-derived smooth muscle cells (AthSMC) and control aortic smooth muscle cells (AoSMC) to various TLR ligands. AthSMC exhibited a specific increase in TLR3 expression and TLR3-dependent functional responses. Intriguingly, exposure to dsRNA in vitro and in vivo induced increased expression of both pro- and anti-inflammatory genes in vascular cells and tissues. Therefore, we sought to assess the contribution of TLR3 signalling in vivo in mechanical and hypercholesterolemia induced arterial injury. Surprisingly, neointima formation in a perivascular collar-induced injury model was reduced by the systemic administration of the dsRNA analogue Poly(I:C) in a TLR3-dependent manner. Furthermore, genetic deletion of TLR3 dramatically enhanced the development of elastic lamina damage after collar-induced injury. Accordingly, deficiency of TLR3 accelerated the onset of atherosclerosis in hypercholesterolemic ApoE^{-/-} mice. Collectively, our data describe for the first time a protective role for TLR signalling in the vessel wall.

### Materials and Methods

Ex Vivo Culture of Cells Isolated from Human Atherosclerotic Plaques

Carotid endarterectomies from patients undergoing surgery for carotid disease were obtained at Charing Cross Hospital, London. The protocol was approved by the Research Ethics Committee RREC2989. All patients gave written informed consent, according to the Human Tissue Act 2004 (UK). Single cell suspensions of mixed cell types were obtained via enzymatic digestion and cultured for 24 hours as previously described (18).

### Isolation and Culture of Atherosclerotic Plaque-Derived Smooth Muscle Cells (AthSMC)

AthSMC were isolated from the mixed atheroma cells via magnetic cell sorting (Miltenyi, MACS), utilizing anti-CD45 (pan-leukocyte marker) and anti-CD31 (endothelial cells) antibodies coupled to microbeads. SMC expressing SMC-alpha actin composed >90% of the cells. Control AoSMC were purchased from Promocell (Germany). Both SMC types were grown in SMC medium (Promocell, Germany) and used at passage 3 consistently in all experiments. The protocols of stimulation with TLR ligands are described below.

### Mice

C57BL/6 mice were purchased from Charles River (Margate, UK). Apolipoprotein E-deficient (ApoE^{-/-}) mice on a C57BL/6 background were bred in house. Toll-like receptor 3-deficient (TLR3^{-/-}) mice fully backcrossed onto a C57BL/6 background were a gift from Richard Flavell (Yale University, New Haven, USA) (21). All mice in this study were male. Animals were housed in specific pathogen free conditions and all experimental animal procedures were approved by the Kennedy Institute of Rheumatology Ethics Committee and performed according to UK Home Office guidelines.

### Perivascular Collar Injury

At 22 weeks of age, male C57BL/6 and TLR3^{-/-} mice were anesthetized with isofluorane by inhalation, the left carotid artery dissected and a non-occlusive tygon collar (length 2.5 mm; internal bore diameter, 510 μm, Cole-Parmer, London, UK) placed around the carotid artery. Mice received 250 μg Poly(I:C) (Sigma, Dorset, UK) or PBS intraperitoneally on alternate days for a total of 8 doses starting 4 days after surgery. Twenty-one days following collar placement, mice were euthanized, and neointima development and the presence of elastic lamina breaks were assessed as described below.

### Analysis of Atherosclerosis Development

ApoE^{-/-}TLR3^{-/-} mice were generated by crossing ApoE^{-/-} mice with TLR3^{-/-} mice. ApoE^{-/-} TLR3^{-/-} double knockout mice were fertile and exhibited no overt phenotype. Mice were fed a standard chow diet and euthanized at either 15 or 30 weeks of age as described below. Aortic root lesion area was assessed as described below.

Stimulation with Cytokines and TLR Ligands

SMC were cultured in 50 cm² tissue culture dishes and grown until near confluence. AthSMC and AoSMC were serum starved for 24 hours and then cultured in DMEM either alone or in the presence of 10 ng/ml IL1a, 100 ng/ml Pam3Cys, 100 ng/ml FSL-1 (Pam2CGDPKHPKSF), 25 μg/ml Poly(I:C), 100 ng/ml Lipopolysaccharide (LPS), 1 μg/ml R837 (Imiquimod) and 1 μg/ml PolyU (all purchased from Invivogen). Cells were serum starved for 24 h prior to stimulation with the indicated agonists. Supernatants were collected 24 hours after stimulation and frozen at −80° C. for batch analysis via ELISA. In the experiments performed on the mixed human atheroma cell culture, cells were cultured immediately after isolation at 10⁶ cells per milliliter in RPMI containing 5% fetal bovine serum (Biosera, UK) in the presence or absence of 25 μg/ml Poly(I:C), 1 μM CpG ODN2006 and 1 μM CpG ODN2006 control (Invivogen) for 24 and 48 hours. The concentrations used were selected on the basis of dose-response experiments as the dose with maximal effect in absence of cell death monitored via MTT.

### ELISA Analysis of Human Cytokine Levels

Cytokine production in supernatants of AthSMC and AoSMC were measured by ELISA using IL-6, IL-8 and CCL2/MCP-1 (Pharmingen, UK). IFNα was detected via a high sensitivity ELISA kit from R&D Systems. Each condition was tested in triplicate and each triplicate was analyzed separately. Concomitantly, viability was monitored with the use of 3-(4,5-dimethyl-2-yl)-2,5-diphenyltetrazolium (MTT) (Sigma, UK).

### Gene Expression Profiling and Quantitative PCR in Human SMCs

SMCs were plated in 9.6 cm² dishes and grown until near confluence. SMCs were serum starved for 24 h prior to stimulation with 25 μg/ml Poly(I:C) for 5 hours. Total cellular RNA was extracted from SMC using RNeasy® Mini Kit (Qiagen) according to the manufacturer's instructions. To remove any residual genomic DNA, RNA samples were treated with DNase (TurboDNase, Ambion) according to manufacturer's instructions. RNA was reverse transcribed to cDNA using M-MLV Reverse Transcriptase (Promega, UK). Quantitative PCR (QPCR) analysis of 84 atherosclerosis related genes was performed using Atherosclerosis RT2 Profiler PCR Arrays (SA Bioscience Corporation, USA) as per the manufacturer's protocol. The complete list of the genes analyzed is available online at http://www.sabiosciences.com/rt_per_product/HTML/PAHS-038A.html. RT2 Profiler PCR arrays were run on an ABI 7900HT machine (Applied Biosystems). Duplicate arrays were run per condition for unstimulated and Poly (I:C)-stimulated AoSMC and AthSMC. Data analysis was performed using the manufacturer's integrated web-based software package for the PCR Array System using Ct based fold-change calculations.

Alternatively, AoSMc and AthSMC were stimulated with 25 μg/ml Poly(I:C) or interferon α (IFN α) at 10 ng/mL or IFNy at 10 ng/mL. Total RNA was extracted as before and TLR3 gene expression was quantified via Q-PCR with TaqMan® Gene Expression Assays (Hs01551078_m1*; Applied Biosystems Inc.).

### Detection of TLR3 Expression on SMC Via Flow Cytometry

SMC were grown until near confluence in 50 cm² tissue culture dishes and serum starved for 24 hours. Cells were then scraped in cold PBS and washed in FACS buffer (PBS in 1 FBS, 0.09% NaN₃). Cells were either left untouched or permeabilized with BD Perm/Wash buffer (BD Biosciences, Oxford, UK). Cells were subsequently stained with FITC-conjugated anti TLR3 antibody or isotype control (Abcam), and analyzed by FACScan (Becton Dickinson) and Flow-Jo Software (TreeStar, USA).

### Real Time—Polymerase Chain Reaction of Murine Tissues

Total RNA was isolated from murine tissues using the Qiagen RNeasy kit (Qiagen, Crawley, UK) according to the manufacturer's instructions. Total RNA was treated with DNase I and reverse-transcribed to cDNA using M-MLV Reverse Transcriptase RNase H-, Point Mutant (Promega, UK) and oligo(dT) primer. RT-PCR was performed using TaqMan Gene Expression Assays (CCL5 (Mm01302427_m1), VCAM1 (Mm01320970_m1), CCL2 (Mm00441242_m1), TLR3 (Mm01207403_m1), IL-10 (Mm01288386_m1), PD-L1 (Mm00452054_m1*), PD-L2 (Mm00451734_m1*) IFNβ (Mm00439552_s1*), (Applied Biosystems) and TaqMan universal PCR Master Mix (Applied Biosystems) on a 7900HT Fast Real-Time PCR System (Applied BioSystems). PCR amplification was carried out for 40 cycles. Samples were normalized to β-actin. The 2-ΔΔCt method was used to analyze the relative changes in gene expression.

### Morphometric Measurement of Neointima Formation in Perivascular Collar Injury

Twenty days following collar placement, mice were euthanized, terminal blood collected via cardiac puncture and the vasculature perfused with 0.9% saline. Injured carotids were dissected out and frozen at −80° C. in Optimal cutting temperature (OCT) compound (ThermoScientific, Runcorn, UK). Sham-operated contralateral arteries were used as controls. Mice were fed regular chow throughout the duration of the experiment.

For the injured carotid artery, serial 5 μm cryosections were taken of the carotid tissue distal to the collar. Five sections were collected on each slide and 15 to 25 slides were collected per arterial segment. The first five alternate slides were stained with Accustain elastic stain kit (Sigma) according to manufacturer's instructions. Measurement of lesion and vessel areas was performed on one section per stained slide using ProgRes CapturePro image analysis software (version 2.5.2.0, Jenoptik, Germany). The area between the internal and external elastic arteries was taken as the medial area and the intimal area was calculated by subtracting the lumen area from the internal elastic lamina area. The intimal medial ratio (IMR) was then calculated by dividing the intimal area by the medial area. The IMR measurements were then averaged for each mouse.

### Analysis of Elastic Lamina Breaks in Perivascular Collar Injury

Elastin-stained slides were also used to assess the integrity of the elastic laminae. The portion of the carotid artery distal to the collar (the same as used for neointima assessment) was divided into 5 segments and representative sections from each segment were evaluated for the presence of interruptions in the elastic lamina. The width of any observed break was measured by drawing a line between the start and end of a break in the external elastic lamina using ProgRes CapturePro image analysis software (version 2.5.2.0, Jenoptik). The width of any elastic lamina break was calculated as the mean width of break across all sections examined for each mouse. Absolute values for size of elastic lamina break were obtained by calibrating the software using an image of a micrometer slide taken at the same magnification.

### Morphometric Measurement of Aortic Root Atherosclerotic Lesion Development

Mice were weaned at 4 weeks of age and fed a standard chow diet for the duration of the experiment. At either 15 or 30 weeks of age, ApoE^{-/-} and ApoE^{-/-}TLR3^{-/-} mice were euthanized with a barbiturate overdose and terminal blood collected from the right ventricle. Hearts were perfused in situ with saline via a cannula inserted into the left ventricle (outflow via an incision in right atria) and then frozen in OCT.

Five micrometer cryosections were taken of the aortic root for the entire region of the valve leaflets and every 20th section (100 μm) was stained with Oil Red O and counterstained with hematoxylin. Aortic root sections were coded and analysed blind. Images were captured under identical microscope, camera and light conditions. Quantification was performed by drawing around the atherosclerotic lesions and the aortic wall using Clemex Vision Lite version 5.0 (Clemex, Longueuil, Canada). Absolute values for cross-sectional area were obtained by calibrating the software using an image of a micrometer slide taken at the same magnification. The individual lesion areas per aortic root section were averaged to obtain the mean lesion area per mouse. The lesion area fraction was calculated by dividing the mean lesion area by the mean area of the aortic wall and expressed as a percentage.

### Immunohistochemistry

Immunohistochemistry was performed on 5 μm cryosections of aortic root sections using standard avidin biotinylated enzyme complex (ABC) methods. In brief, sections were fixed in ice-cold acetone before incubation with 10% normal rabbit or goat serum for one hour. Following a wash in PBS, endogenous avidin and biotin were blocked using Vector avidin/biotin blocking kit (Vector labs, Peterborough, UK) according to manufacturer's instructions. Sections were then incubated with a primary antibody against CD68 for macrophages (clone FA-11; AbD Serotec, Oxford, UK) for 45 minutes at room temperature followed by relevant biotinylated secondary antibodies. Following blocking of endogenous peroxidase activity with 0.3% hydrogen peroxide, sections were incubated with avidin and biotinylated horseradish peroxidase macromolecular complexes using Vectastain Elite ABC kit (Vector Labs) according to manufacturer's instructions. Bound peroxidise was detected using 3,3′-diaminobenzidine (DAB) and nuclei counterstained with hematoxylin. Staining using an appropriate isotype-matched control was performed on a consecutive section as a control.

### Masson Trichrome Staining

Masson trichrome staining was performed using standard staining protocols. In brief, slides were incubated for 1 hour in 5% chromic acid before a 4 minute incubation in Celestine blue. After 4 minutes in Harris haematoxylin, slides were then dipped briefly in acid alcohol before a 5 minute incubation in ponceau red/acid fuchsin solution. Following 30 seconds in 1% phosphomolybdic acid and a 3 minute incubation in 1% fast green solution, slides were dehydrated and coverslipped.

### Quantification of Immunohistochemical and Masson Trichrome Staining

Aortic root lesion area staining positive for CD68 (brown staining) or collagen (green staining) was quantified using Clemex Vision Lite version 5.0. Images were captured under identical microscope, camera and light conditions, coded and analysed blind. Using the image analysis software, positive staining was detected and lesion area measured. Absolute values were obtained by calibrating the software using an image of a micrometer slide taken at the same magnification. Lesion area fraction staining positive for CD68 or collagen was calculated by dividing the area positive by the lesion area and expressing it as a percentage.

### Serum Cholesterol Quantification

Total serum cholesterol levels in ApoE−/− and ApoE−/−TLR3−/− mice were determined using a Cholesterol/Cholesteryl Ester Quantitation Kit (BioVision, California, USA) according to manufacturer's instructions.

### Statistical Analysis

Data was analyzed with STATA (Version 10, StateCorp LP, Texas, USA) or GraphPad Prism (version 5.02, La Jolla, USA) as appropriate. All data are expressed as Mean±SEM unless otherwise stated. Rank analysis of covariance (rank ANCOVA) was used to assess the effect of treatment of cells in culture in order to take into account the effect of baseline cytokine production. For data that passed a normality test, student's t-tests or One-way analysis of variance with Dunnett's multiple comparison test were used as appropriate. Where data did not pass a normality test, Mann-Whitney U tests were performed. Chi-square tests were also performed as appropriate. An alpha level of 0.05 was considered as statistically significant. All tests used were 2-tailed.

### Results

### Increased Expression of TLR3 by Atheroma-Derived SMCs (AthSMC) and its Mechanism.

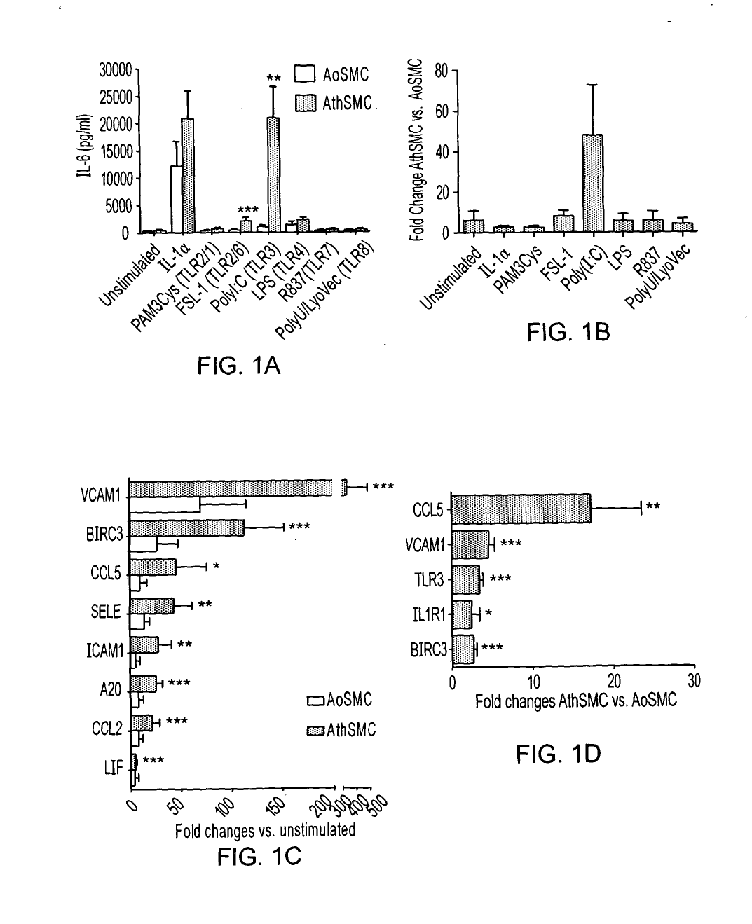

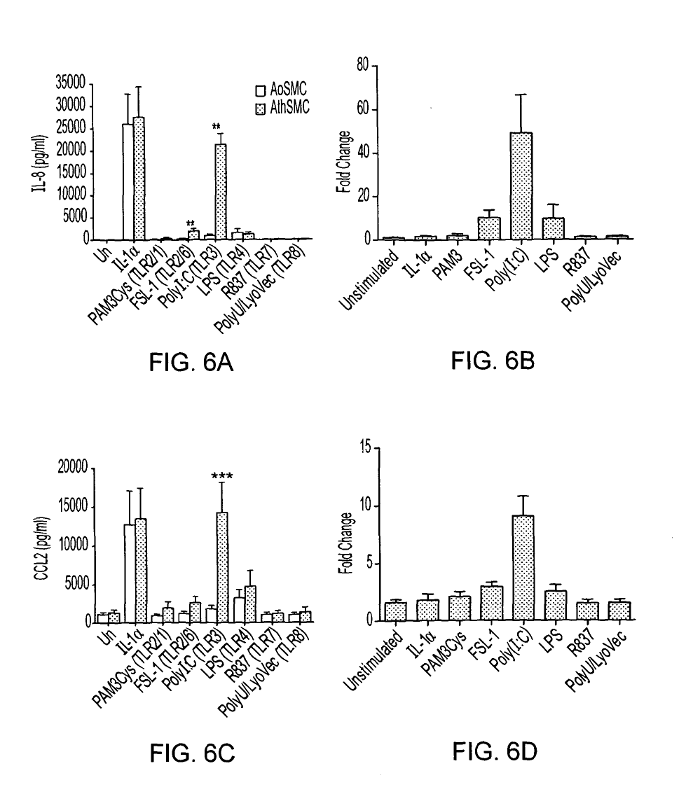

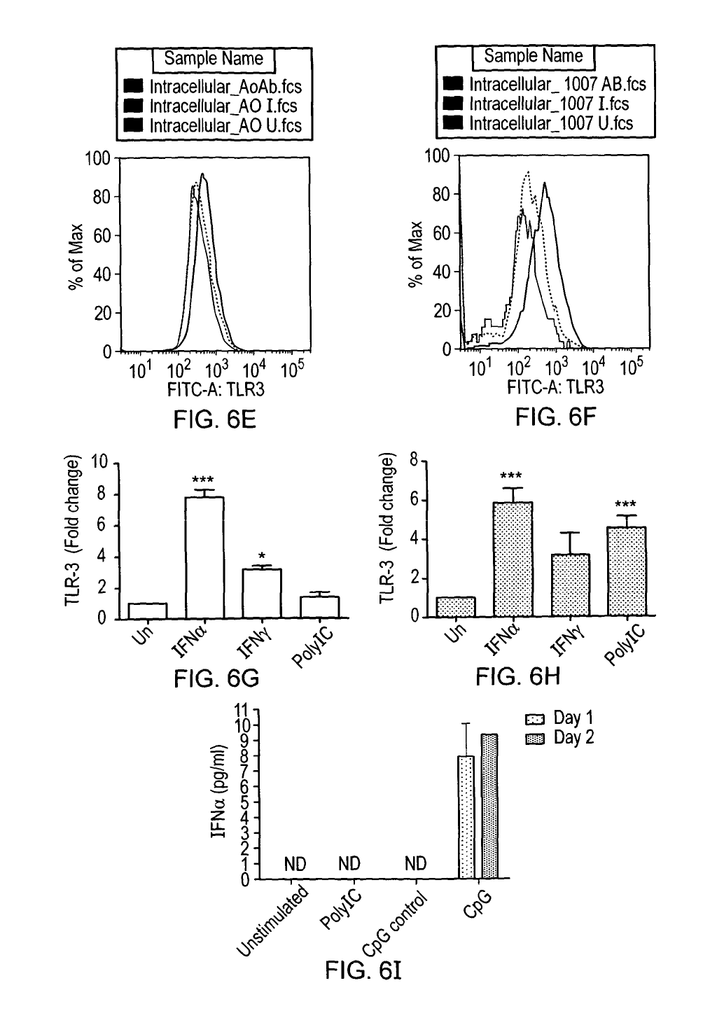

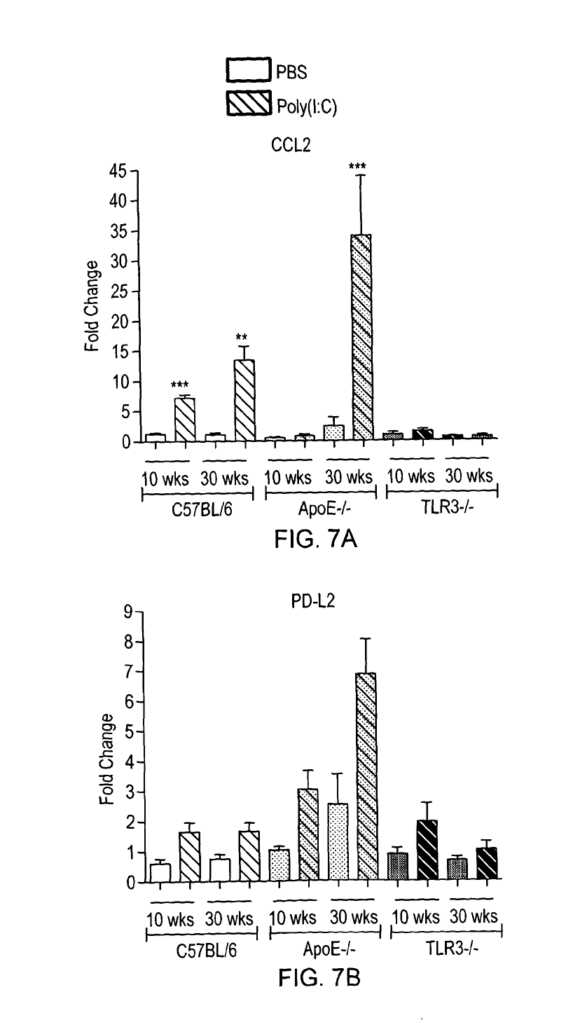

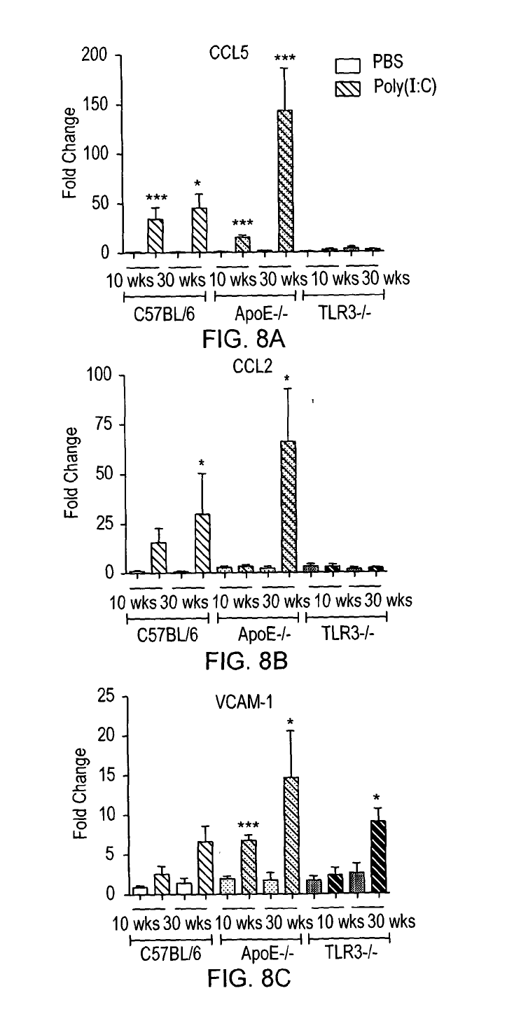

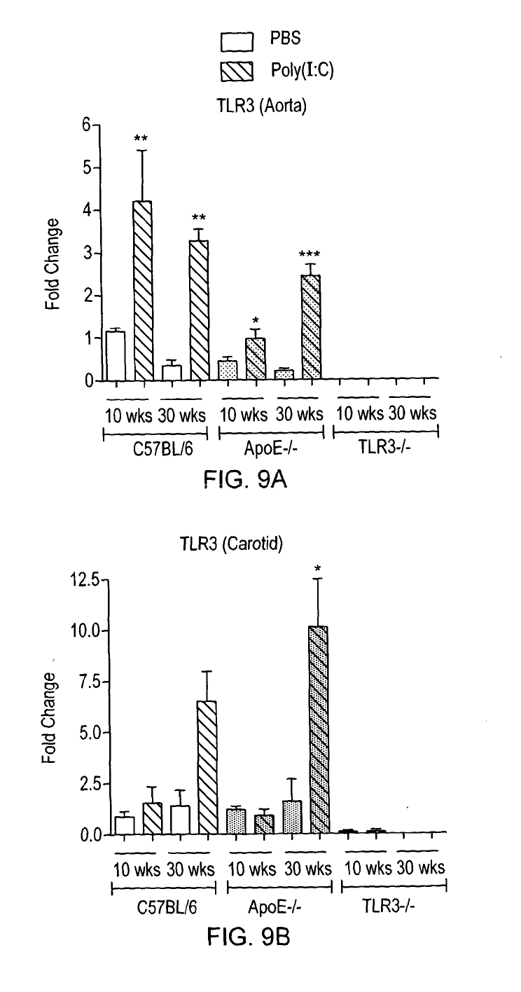

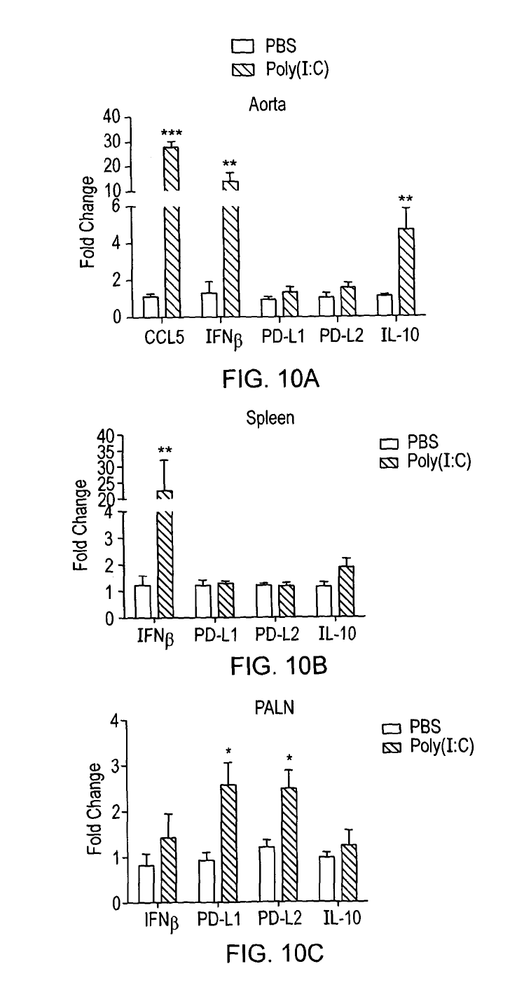

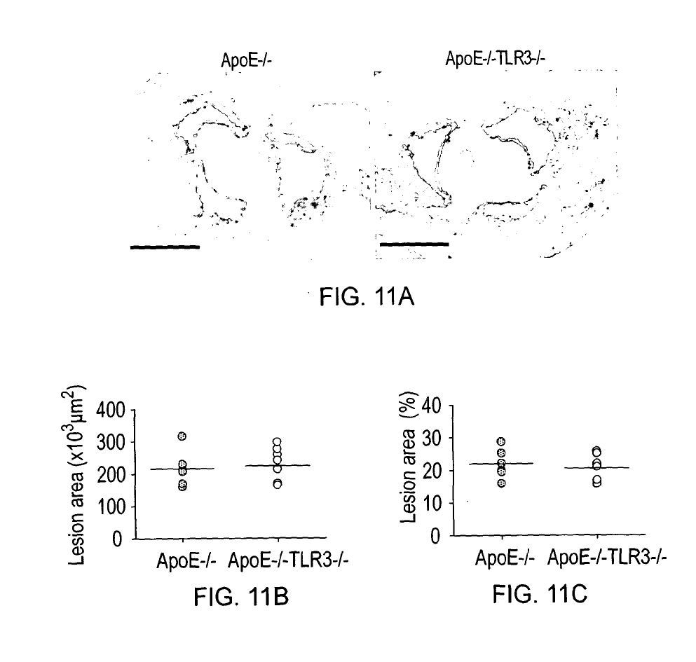

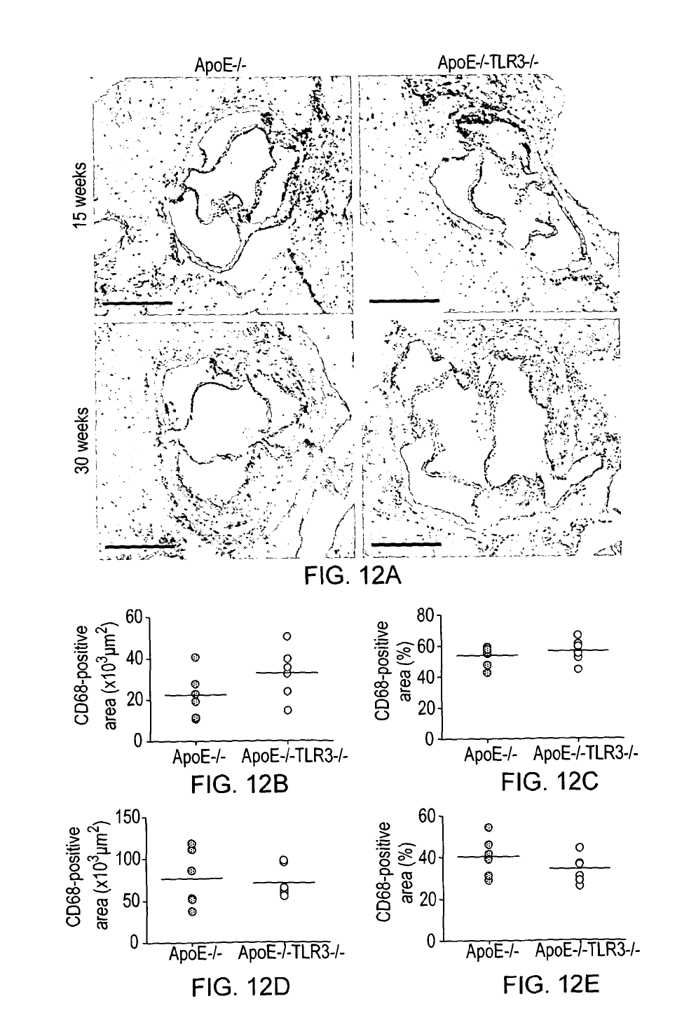

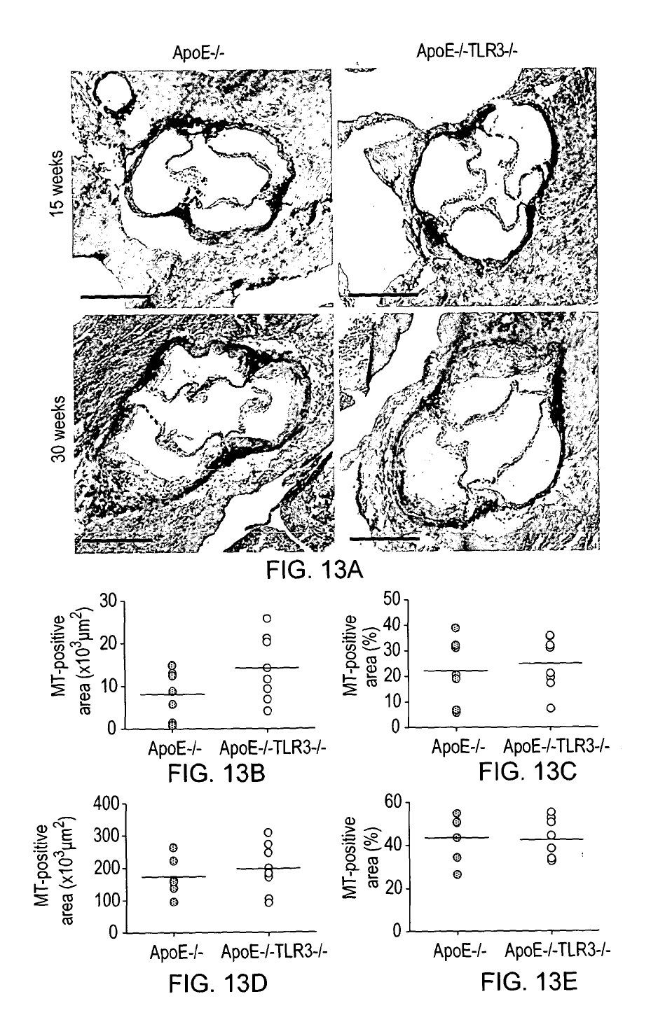

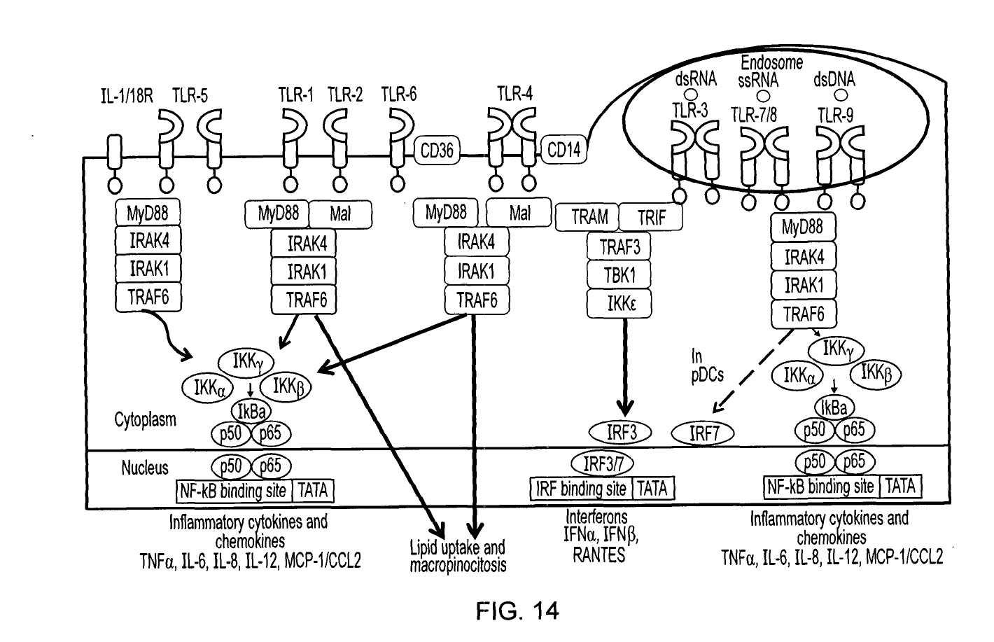

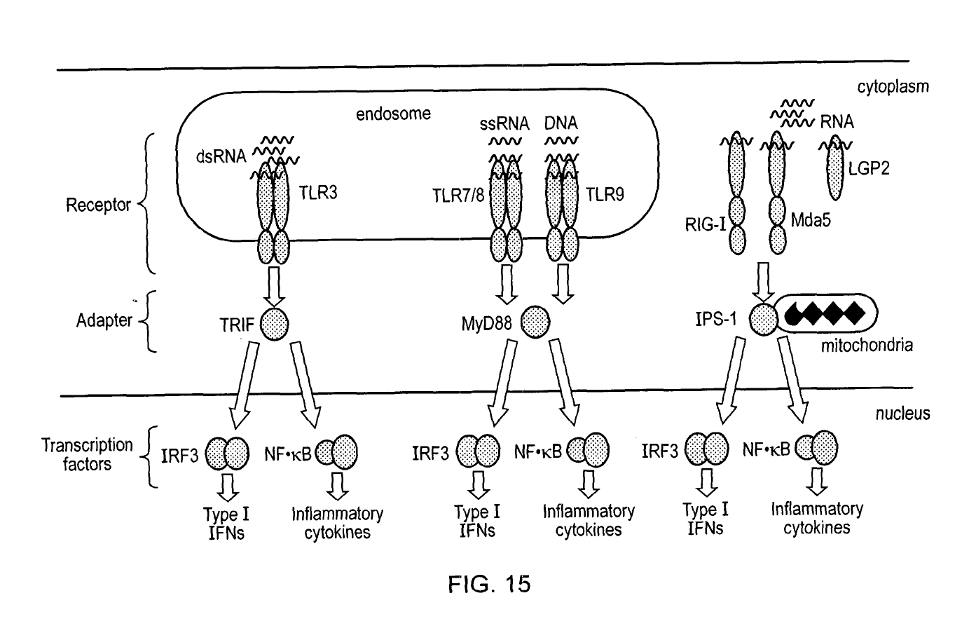

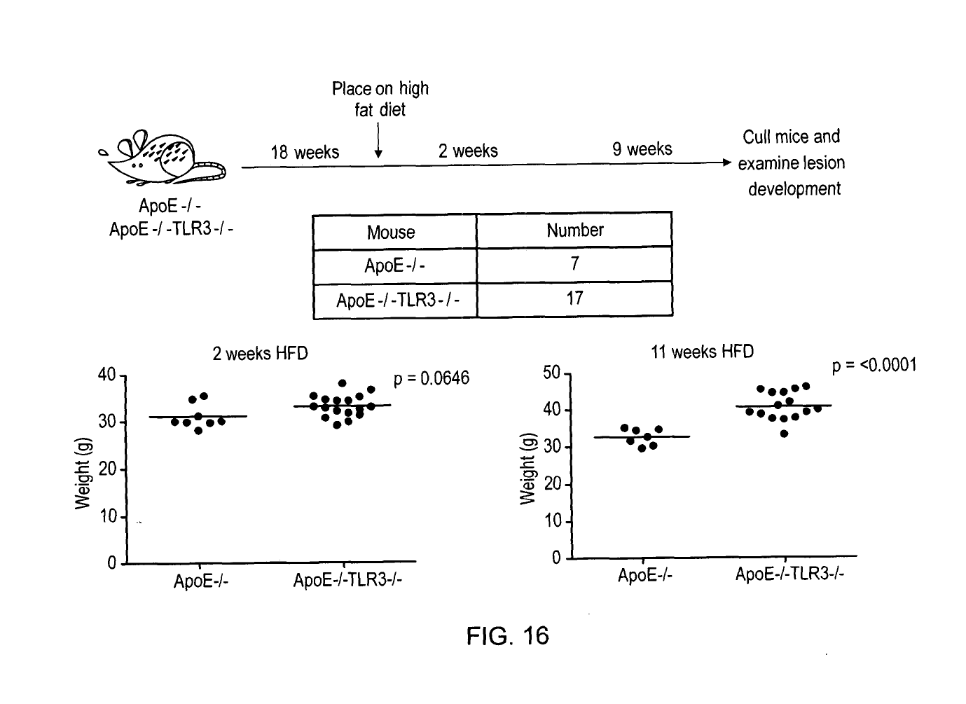

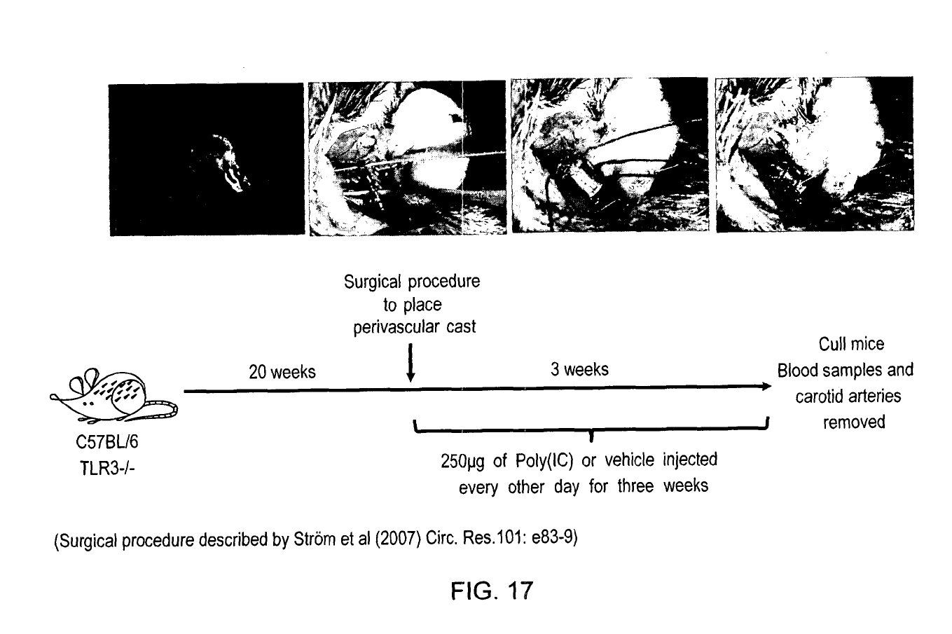

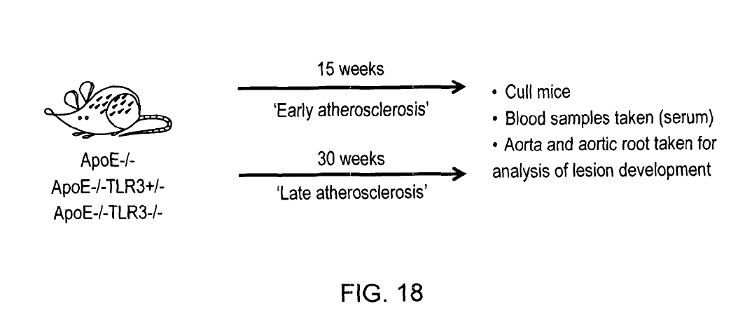

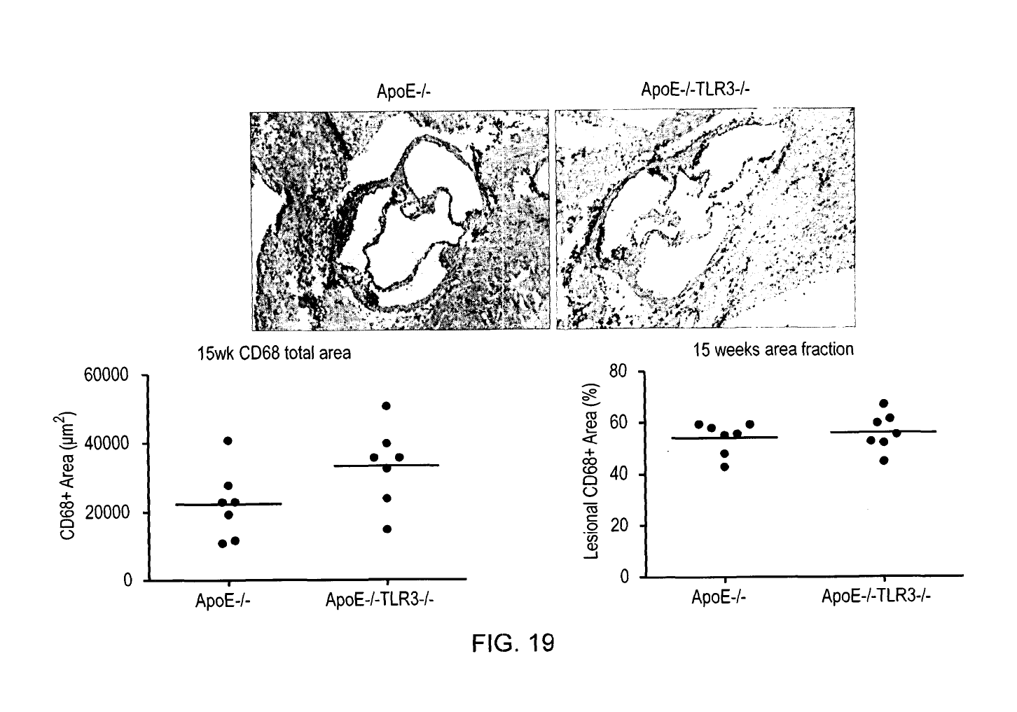

We screened responses to TLR-IL-1 family agonists in AthSMC and control aortic SMC (AoSMC). SMC have been previously shown to respond to a variety of TLR agonists (17). However, AthSMC specifically displayed an enhanced expression of IL-6, IL-8 and CCL2/MCP-1 when stimulated with the dsRNA synthetic analogue Poly(I:C) compared to AoSMC (FIGS. 1A&B and FIG. 6A-D). To a lesser extent, the TLR2/TLR6 agonist FSL-1 induced an enhanced response in AthSMC vs. AoSMC in terms of IL-6 and IL-8 but not CCL-2/MCP-1 production. Other TLR agonists did not elicit enhanced responses in AthSMC vs. AoSMC. As the TLR3 dependent responses in AthSMC were particularly marked (over 40-fold compared to AoSMC; FIG. 1B), we explored further the gene expression of atherosclerosis-relevant genes by Q-PCR array (SABiosciences). dsRNA stimulation significantly increased the expression of a selected set of genes involved in inflammatory cell recruitment (e.g VCAM1 and CCL5) and regulation of inflammation (e.g. A20 and BIRC3/cIAP) in AthSMC, but not in AoSMC (FIG. 1C).

In order to assess the mechanism of the increased response to the dsRNA analogue Poly(I:C) of AthSMC, we compared the baseline gene expression of AthSMC and AoSMC in unstimulated conditions. TLR3 expression was 3.4-fold higher in AthSMC vs. AoSMC (FIG. 1D). Upregulation of TLR3 protein was verified by FACS analysis (FIGS. 6E&F). Interestingly, genes that were found to be upregulated by dsRNA stimulation such as VCAM-1, BIRC3/c-IAP2 and CCL5/RANTES, were also significantly higher in AthSMC vs. AoSMC even in unstimulated condition, suggesting prior in vivo TLR stimulation. Hence the mechanism of the increased dsRNA responsiveness in AthSMC appears to be an upregulation of TLR3.

We hypothesized that exposure of AthSMC to interferons (IFN) within the atherosclerotic plaque was a potential mechanism of upregulation of TLR3. To test this hypothesis, we exposed both AoSMC and AthSMC to IFNγ and IFNα and quantified TLR3 gene expression by Q-PCR. IFNα, and to a lesser extent IFNγ, were able to increase the expression of TLR3 (FIGS. 6G&H). Interestingly, Poly(I:C) itself is able to upregulate the expression of TLR3 selectively in AthSMC but not in AoSMC (FIGS. 6G&H), which is consistent with our previous findings. We have previously shown that a mixed cell population of cells isolated from human atheroma is able to spontaneously produce TNF, IL-1 and IFNγ (18). However, we were unable to detect IFNα. Earlier work by Weyand's group demonstrated IFNα production after TLR9 stimulation with CPG DNA in human atherosclerotic plaques (19). We confirmed in our mixed cell type atheroma cell culture system that IFNα production could be induced by TLR9 stimulation (FIG. 61).

Therefore, we demonstrate that SMC isolated from human carotid atheroma have an increased responsiveness to dsRNA due to increased TLR3 expression. Type I interferons produced in the atherosclerotic plaque contribute to this increased TLR3 expression.

### Augmented Gene Expression of Pro- and Anti-Inflammatory Genes Following In Vivo TLR3 Activation.

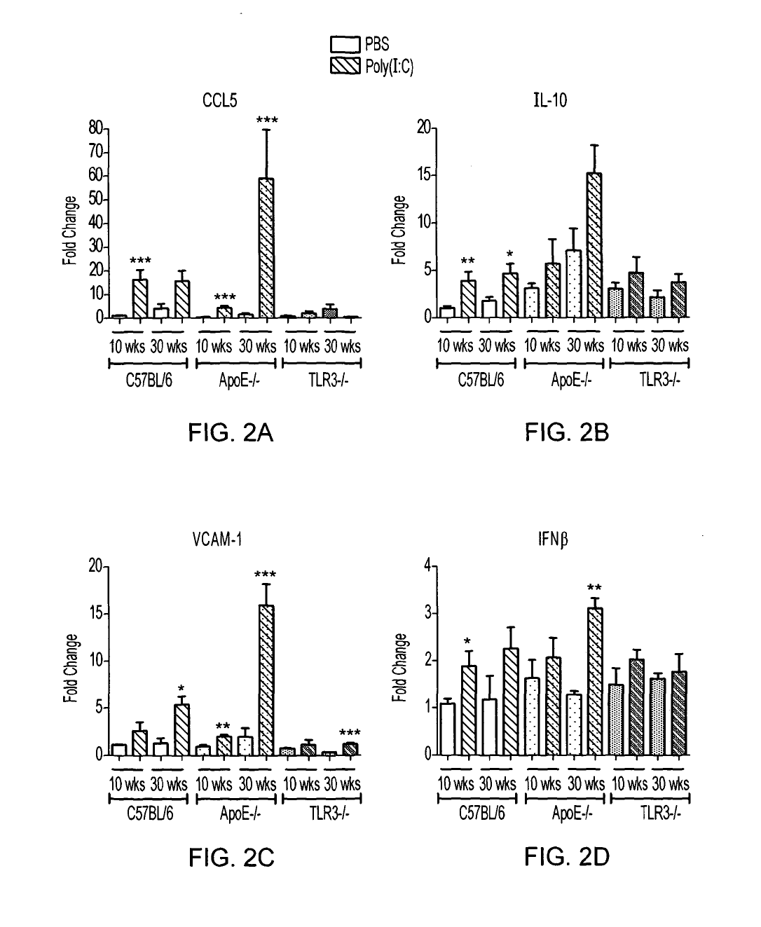

In order to investigate whether the increased TLR3 expression in AthSMC vs. AoSMC was due to the different arterial site (carotid vs. aorta), or presence or absence of atherosclerotic disease, we sought to examine the effect of i.p. Poly(I:C) stimulation in vivo in 10- and 30-week old C57BL/6, ApoE^{-/-} and TLR3^{-/-} mice. In accordance with our in vitro data, stimulation with a single dose of Poly(I:C) induced the aortic expression of both pro-inflammatory genes including CCL5/RANTES, CCL2/MCP-1 and VCAM-1 (FIGS. 2A&C and FIG. 7A) and anti-inflammatory genes such as IFNβ, IL-10 and PD-L2 (FIGS. 2B&D and FIG. 7B). A20 induction was not observed in mice. This effect was more pronounced in the aorta of ApoE^{-/-} mice with advanced disease compared to young ApoE^{-/-}. The effect of Poly(I:C) stimulation was even more marked in carotid artery tissue indicating that the human AthSMC responses might reflect both disease development and arterial site (FIG. 8). The expression of these genes was largely dependent on TLR3 and almost abrogated in TLR3^{-/-} mice. Poly(I:C) up-regulated TLR3 gene expression in vivo in vascular tissues (FIG. 9), which also fits with our in vitro human data. Expression of selected genes in aortas and secondary lymphoid organs of C57BL/6 mice that received chronic Poly(I:C) stimulation for 3 weeks was also examined. In the aorta of Poly(I:C) treated C57BL/6 mice, expression of CCL5, IFNβ and IL10 was significantly increased (FIG. 10A). Expression of IFNβ mRNA was also induced in the spleen of Poly(I:C) treated C57BL/6 mice (FIG. 10B). In addition, gene expression of PD-L1 and PD-L2 were induced by Poly(I:C) stimulation in the para-aortic lymph nodes (FIG. 10C). These results suggest that TLR3 activation induces gene expression of both potentially detrimental and protective genes.

### Therapeutic Effect of Poly(I:C) and TLR3 on Neointima Formation in Response to Arterial Injury.

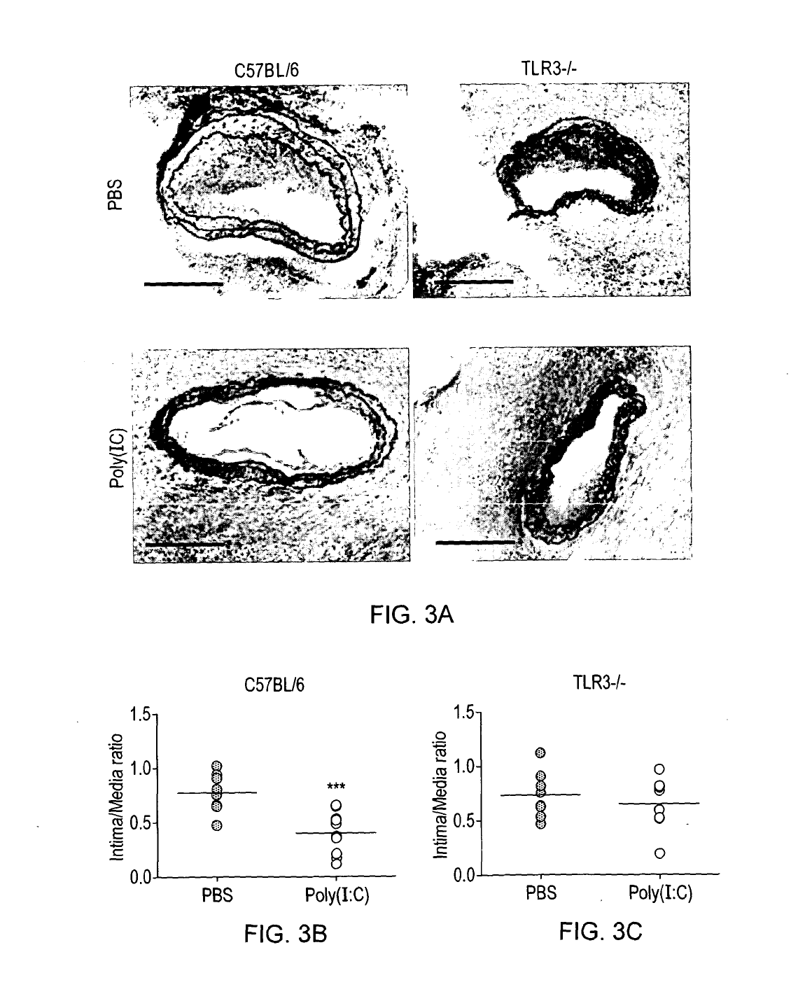

To determine the role of TLR3 activation on injury-induced neointima formation, we utilized a well characterized arterial injury model involving the placement of a perivascular collar, based on an earlier rabbit model first described by Salvador Moncada (20) in C57BL/6 and TLR3−/− mice (21). After collar placement, the mice were treated either with Poly(I:C) or vehicle alone three times a week for 3 weeks. No difference between the end-weight of all groups of mice was observed. Neointima formation upon collar placement, assessed by intima/media ratio, was significantly reduced in Poly(I:C)-treated C57BL/6 mice compared to vehicle-treated mice (p<0.001) (FIG. 3B). Protection against neointima formation after Poly(I:C) treatment was ablated in TLR3^{-/-} mice (p>0.05) (FIG. 3C), indicating that the protective effect of the dsRNA analogue was mediated by TLR3.

### Genetic Deletion of TLR3 Enhances Elastic Lamina Damage Upon Arterial Injury.

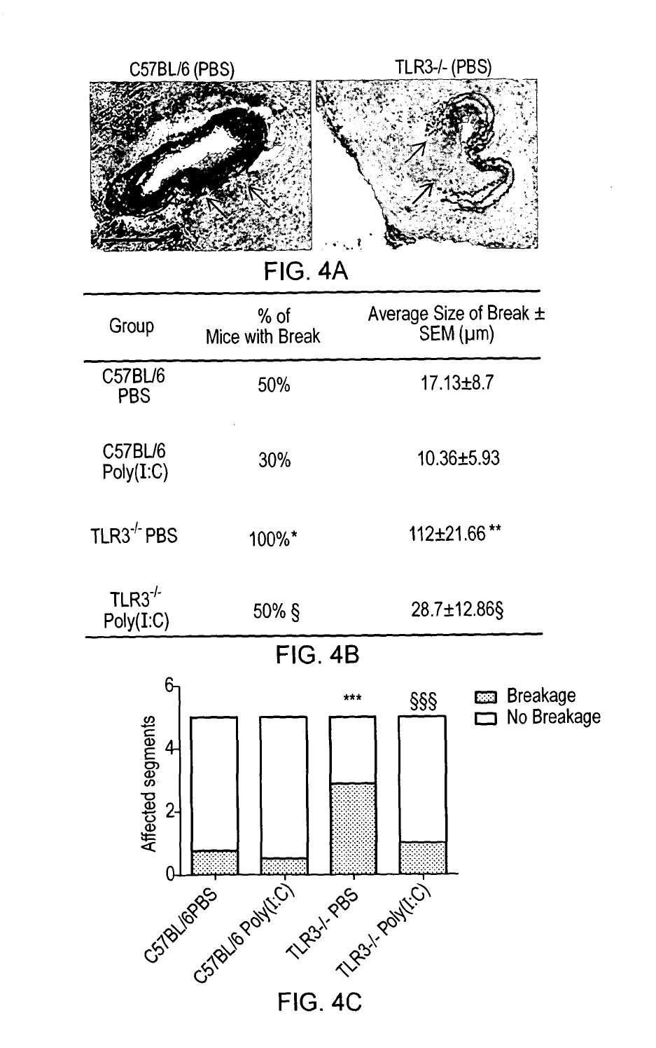

Upon collar placement, short interruptions of the elastic lamina were occasionally observed in elastin Van Gieson-stained tissue sections in C57BL/6 mice (FIG. 4A). In contrast, all TLR3^{-/-} mice developed significantly larger interruptions of the elastic laminas after collar placement (FIG. 4). The interruptions in the elastic lamina were located immediately beneath the neointimal lesion and were not present in contralateral arteries. In comparison to C57BL/6 mice, the interruptions to the elastic laminas were more frequent (FIG. 4B), had a significantly larger cross-sectional width (FIGS. 4A and 4B), and longitudinal span across the carotid artery in TLR3^{-/-} mice versus C57BL/6 wild-type mice (FIG. 4C). These findings indicate a novel role for TLR3 in vasculoprotective mechanisms at the level of the medial layer of the artery. Systemic administration of Poly(I:C) reduced the frequency, depth and width of the elastic lamina breaks in TLR3^{-/-} mice (FIGS. 4B&C), suggesting that in the absence of TLR3, other dsRNA receptors can mediate protection.

### TLR3 Deficiency Accelerates Early Atherosclerosis in Hyperlipidemic Mice.

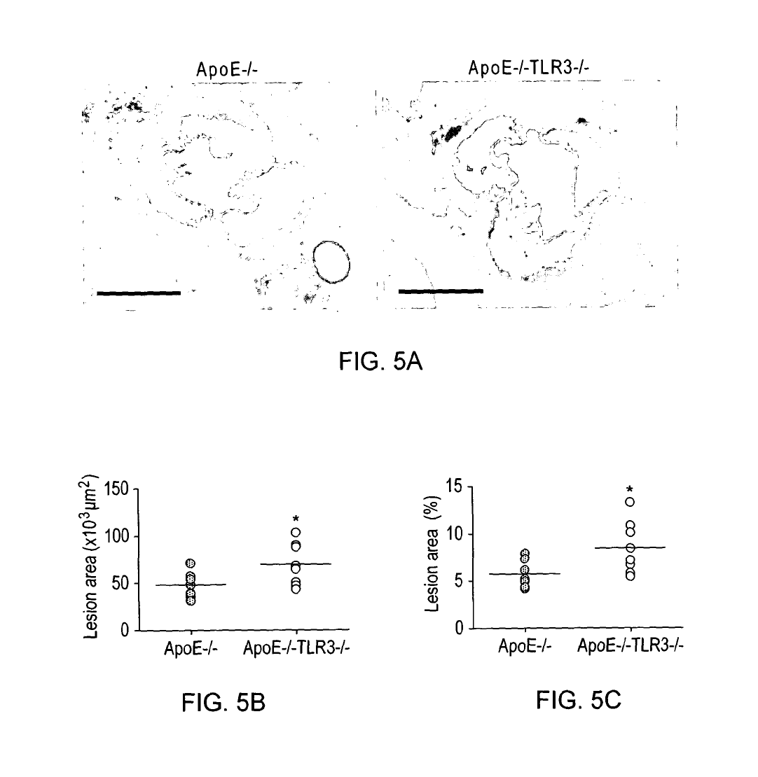

To assess the effect of TLR3 deficiency on atherosclerotic lesion development, ApoE^{-/-} mice were crossed with TLR3^{-/-} mice to generate ApoE^{-/-}TLR3^{-/-} mice. ApoE^{-/-} and ApoE^{-/-} TLR3^{-/-} mice were fed a normal chow diet and were culled at 15- or 30-weeks of age. No difference in body weight or total serum cholesterol levels was observed between ApoE^{-/-} and ApoE^{-/-}TLR3^{-/-} mice at either time point examined. Fifteen-week ApoE^{-/-}TLR3^{-/-} mice displayed a greater than 40% increase in aortic root atherosclerotic lesion formation compared to ApoE^{-/-} mice with both absolute lesion size and lesion area fraction being significantly increased (p<0.05; FIG. 5). However, no difference in absolute aortic root lesion size or lesion area fraction at the aortic root was observed between ApoE^{-/-} and ApoE^{-/-}TLR3^{-/-} mice aged 30 weeks (FIG. 11).

Composition of atherosclerotic lesions in ApoE^{-/-} and ApoE^{-/-}TLR3^{-/-} mice was assessed by performing CD68-staining to visualize plaque macrophages and Masson trichrome staining to identify collagen. Although a trend towards increased lesional macrophage content in 15-week ApoE^{-/-}TLR3^{-/-} versus ApoE^{-/-} mice was observed statistical significance was not reached (p>0.05; FIG. 12B). However, when differences in lesion area were taken into account, no difference in lesion macrophage content between ApoE^{-/-} and ApoE^{-/-}TLR3^{-/-} mice was observed (FIG. 12C). Furthermore, both absolute lesional CD68-positive area and lesion area fraction staining positive for CD68 were similar in 30-week ApoE^{-/-} and ApoE^{-/-}TLR3^{-/-} mice (FIGS. 12D&E). In 15-week ApoE^{-/-} and ApoE^{-/-}TLR3^{-/-} aortic root lesions, limited collagen was observed and there was no difference in lesional collagen content between the 2 strains (FIGS. 13B&C). In addition, no difference in absolute lesion collagen content or lesion area fraction collagen content was observed between 30-week ApoE^{-/-} and ApoE^{-/-}TLR3^{-/-} mice (FIGS. 13D&E).

### DISCUSSION

Toll like receptors (TLRs) are among the oldest components of the innate immune system, their homologues existing in Drosophila (22). The list of endogenous TLR ligands is growing. Endogenous TLR ligands appear to promote disease, e.g. Immunoglobulins/DNA complexes in lupus via TLR9 (5), extracellular matrix proteins such as tenascin C in arthritis via TLR4 (23) and modified LDL in atherosclerosis via TLR4 (24). Our own work on a unique human carotid atherosclerosis cell extraction and culture system revealed that TLR2 is markedly pro-atherogenic. TLR2 blockade reduces both cytokines and destructive matrix metalloproteinase enzymes, suggesting that a—yet to be identified—TLR2 agonist may be present in human atherosclerotic plaques and it may be a useful therapeutic target (25).

Herein, we report that vascular SMC isolated from human atherosclerotic tissue highly express TLR3 and are primed for TLR3-dependent gene expression via dsRNA. Intriguingly, TLR3 was capable of inducing both pro-inflammatory and anti-inflammatory responses in the vessel wall in vitro and in vivo. Surprisingly, the net effect of TLR3 signalling is protective in in vivo models of mechanical as well as hypercholesterolemic arterial injury. This is the first documentation of TLR-mediated protection in this major human disease process.

Heterogeneity of TLR expression in arterial vessels has been reported previously, yet the functional significance of TLR expression was not studied in the context of disease (26, 27). We took advantage of comparing SMC from disease site (AthSMC) with control aortic SMC (AoSMC) and we noted a dramatic and functional increase of TLR3 expression in AthSMC. Augmented expression of TLR3 has been previously found in diseased tissue in other models, e.g. rheumatoid arthritis (28) and sepsis (29). TLR3 expression in SMC was upregulated by type I and II interferons and Poly(I:C), a synthetic analogue of dsRNA. Poly(I:C) was also able to upregulate TLR3 expression in vascular tissue in vivo, mirroring the in vitro data in cultured SMC. As viral genome-dependent induction of TLR3 is blocked by neutralizing type I interferons or their receptor (30), Poly(I:C)-induced upregulation of TLR3 in our systems may also be due to autocrine IFN signalling.

The effect of the dsRNA analogue on AthSMC was dramatic compared to that in AoSMC, and it induced expression of genes involved in cell recruitment and inflammation (e.g. VCAM-1, CCL2/MCP-1 and CCL5/RANTES). It is noteworthy that anti-inflammatory and anti-apoptotic genes (e.g. A20 and BIRC3) were also upregulated. Similarly, Poly(I:C) administration induced both pro-inflammatory (e.g. CCL5/RANTES, CCL2/MCP-1 and VCAM-1) and anti-inflammatory genes (e.g. IL-10 and PD-L2) in the vascular tissues of both ApoE −/− and wild-type mice. In keeping with the human SMC in vitro studies, the effect of TLR3 was more dramatic in aortas of mice with atherosclerotic disease.

Following these observations, an important question was: what is the net effect of TLR3 signalling in vivo? Systemic delivery of dsRNA prevented neointima formation after placement of a perivascular collar in a TLR3-dependent manner. Moreover, large interruptions in the elastic lamina were induced by the collar in all TLR3^{-/-} mice, revealing an endogenous protective role for TLR3 in vessel wall integrity upon mechanical injury. Treatment with Poly(I:C) reduced the occurrence of the elastic lamina breaks in TLR3^{-/-} mice, suggesting that—in absence of TLR3—other sensors of dsRNA such as melanoma differentiation-associated protein 5 (MDA5) can mediate protection (4). Finally, TLR3 deficiency resulted in the accelerated onset of atherosclerosis in ApoE^{-/-} mice, implying a role for TLR3 in protection from hypercholesterolemic arterial injury. Collectively, our data indicate a role for TLR3 in vessel wall integrity.

Moreover, by showing acceleration of atherosclerosis development and enhanced elastic lamina damage in the absence of TLR3 and an exogenous viral stimulus, we implicate endogenous agonists—yet to be discovered—in vascular protection. TLR3 senses dsRNA in the endosome, a replication by-product of viral replication. However, TLR3 has been increasingly linked to tissue damage. Endogenous RNA released by damaged tissue or necrotic cells is able to induce TLR3 expression and signalling (31), while the alarmin high-mobility group protein B1 sensitizes TLR3 to the recognition of RNA (32). Interestingly, stathmin, a protein with regulatory function on microtubule assembly that is upregulated in brain injury, has been described as a candidate TLR3 agonist, linked to the induction of a neuroprotective gene profile (33). However, no study has so far assigned a clear protective role to TLR3 endogenous agonists in disease.

In the past, there has been speculation about the pathogenic role of viruses in atherosclerotic type lesions e.g. in chicken (34). The pathogenic viral mechanisms reported include cytolytic (35) and immunomediated effects (36). Our study shows that such mechanisms are clearly distinct from the effect of dsRNA and its sensor/s, which—on their own—exert protection. The molecular mechanism(s) of TLR3/Poly(I:C) induced protection are not yet fully unravelled. The dsRNA motif induces IFNβ via TLR3 and MDA5. IFNβ is therapeutic in some, but not all, patients with multiple sclerosis. The mechanism of such heterogeneity in therapeutic response has been partially elucidated by Lawrence Steinman, who showed effectiveness of IFN in Th1/IFNγ—dependent but not Th17/IL-17-dependent EAE (37). Whether the vasculoprotection provided by TLR3 is dependent on the production of type I IFNs is uncertain, as IFNβ therapy has shown conflicting results in animal models of atherosclerosis (38, 39). The production of protective mediators, including IL-10, following TLR3 activation has been reported (40). In our study TLR3 increased expression of IL-10 in vascular tissues, suggesting that IL-10, a cytokine beneficial in various disease models, including atherosclerosis could mediate protection. The B7 family members PDL1 and PDL2, which are augmented after TLR3 stimulation may also contribute to vascular protection (41, 42).

This study enhances our knowledge of the complex role of TLRs in health and disease and points to therapeutic opportunities. Although it is unlikely that Poly(I:C) itself is a candidate due to unsuitable pharmacology, unravelling the pathways of protection might permit the development of novel therapeutics for the treatment of cardiovascular disease. In contrast to current concepts, TLRs are not always detrimental in vascular disease (7, 10, 25) but they can be relevant in repair mechanisms within the vessel wall. It also suggests a new paradigm: might we do better therapeutically by enhancing natural homeostatic regulatory pathways than by blocking putative pathogenic ones?

### REFERENCES

1. Lopez A D, Mathers C D, Ezzati M, Jamison D T, & Murray C J (2006) Global and regional burden of disease and risk factors, 2001: systematic analysis of population health data. Lancet 367(9524):1747-1757.
2. Montecucco F & Mach F (2009) Update on statin-mediated anti-inflammatory activities in atherosclerosis. Semin Immunopathol 31(1):127-142.
3. Medzhitov R & Janeway C A (1997) Innate immunity: the virtues of a nonclonal system of recognition. Cell 91(3):295-298.
4. Takeuchi O & Akira S (2010) Pattern recognition receptors and inflammation. Cell 140(6):805-820.
5. Leadbetter E, at al. (2002) Chromatin-IgG complexes activate B cells by dual engagement of IgM and Toll-like receptors. Nature 416(6881):603-607.
6. Bjorkbacka H, at al. (2004) Reduced atherosclerosis in MyD88-null mice links elevated serum cholesterol levels to activation of innate immunity signaling pathways. Nat Med 10(4):416-421.
7. Michelsen K S, et al. (2004) Lack of Toll-like receptor 4 or myeloid differentiation factor 88 reduces atherosclerosis and alters plaque phenotype in mice deficient in apolipoprotein E. Proc Natl Acad Sci USA 101(29):10679-10684.
8. Prinz M, et al. (2006) Innate immunity mediated by TLR9 modulates pathogenicity in an animal model of multiple sclerosis. J Clin Invest 116(2):456-464.
9. Choe J Y, Crain B, Wu S R, & Corr M (2003) Interleukin 1 receptor dependence of serum transferred arthritis can be circumvented by toll-like receptor 4 signaling. J Exp Med 197(4):537-542.
10. Mullick A, Tobias P, & Curtiss L (2005) Modulation of atherosclerosis in mice by Toll-like receptor 2. J Clin Invest 115(11):3149-3156.
11. Aumeunier A, et al. (2010) Systemic Toll-like receptor stimulation suppresses experimental allergic asthma and autoimmune diabetes in NOD mice. PLoS One 5(7):e11484.
12. Marta M, Andersson A, lsaksson M, Kampe O, & Lobell A (2008) Unexpected regulatory roles of TLR4 and TLR9 in experimental autoimmune encephalomyelitis. Eur J Immunol 38(2):565-575.
13. Rachmilewitz D, at al. (2002) Immunostimulatory DNA ameliorates experimental and spontaneous murine colitis. Gastroenterology 122(5):1428-1441.
14. Vijay-Kumar M, et al. (2007) Deletion of TLR5 results in spontaneous colitis in mice. J Clin Invest 117(12):3909-3921.
15. Vijay-Kumar M, et al. (2007) Activation of toll-like receptor 3 protects against DSS-induced acute colitis. Inflamm Bowel Dis 13(7):856-864.
16. Mullick A E, et al. (2008) Increased endothelial expression of Toll-like receptor 2 at sites of disturbed blood flow exacerbates early atherogenic events. J Exp Med 205(2):373-383.
17. Yang X, et al. (2006) Toll-like receptor 3 signaling evokes a proinflammatory and proliferative phenotype in human vascular smooth muscle cells. Am J Physiol Heart Circ Physiol 291(5): H2334-2343.
18. Monaco C, et al. (2004) Canonical pathway of nuclear factor kappa B activation selectively regulates proinflammatory and prothrombotic responses in human atherosclerosis. Proc Natl Acad Sci USA 101(15):5634-5639.
19. Niessner A, et al. (2006) Pathogen-sensing plasmacytoid dendritic cells stimulate cytotoxic T-cell function in the atherosclerotic plaque through interferon-alpha. Circulation 114 (23): 2482-2489.
20. Booth R F, et al. (1989) Rapid development of atherosclerotic lesions in the rabbit carotid artery induced by perivascular manipulation. Atherosclerosis 76(2-3):257-268.
21. Alexopoulou L, Holt A C, Medzhitov R, & Flavell R A (2001) Recognition of double-stranded RNA and activation of NF-kappaB by Toll-like receptor 3. Nature 413(6857):732-738.
22. Lemaitre B, Nicolas E, Michaut L, Reichhart J M, & Hoffmann J A (1996) The dorsoventral regulatory gene cassette spatzle/Toll/cactus controls the potent antifungal response in Drosophila adults. Cell 86(6):973-983.
23. Midwood K, et al. (2009) Tenascin-C is an endogenous activator of Toll-like receptor 4 that is essential for maintaining inflammation in arthritic joint disease. Nat Med 15(7):774-780.
24. Stewart C R, et al. (2010) CD36 ligands promote sterile inflammation through assembly of a Toll-like receptor 4 and 6 heterodimer. Nat Immunol 11(2):155-161.
25. Monaco C, et al. (2009) Toll-like receptor-2 mediates inflammation and matrix degradation in human atherosclerosis. Circulation 120(24):2462-2469.
26. Pryshchep O, Ma-Krupa W, Younge B R, Goronzy J J, & Weyand C M (2008) Vessel-specific Toll-like receptor profiles in human medium and large arteries. Circulation 118(12): 1276-1284.
27. Ahmad U, et al. (2010) IFN-gamma primes intact human coronary arteries and cultured coronary smooth muscle cells to double-stranded RNA- and self-RNA-induced inflammatory responses by upregulating TLR3 and melanoma differentiation-associated gene 5. J Immunol 185(2):1283-1294.
28. Roelofs M F, et al. (2005) The expression of toll-like receptors 3 and 7 in rheumatoid arthritis synovium is increased and costimulation of toll-like receptors 3, 4, and 7/8 results in synergistic cytokine production by dendritic cells. Arthritis Rheum 52(8):2313-2322.
29. Cavassani K A, et al. (2008) TLR3 is an endogenous sensor of tissue necrosis during acute inflammatory events. J Exp Med 205(11):2609-2621.
30. Miettinen M, Sareneva T, Julkunen I, & Matikainen S (2001) IFNs activate toll-like receptor gene expression in viral infections. Genes Immun 2(6):349-355.
31. Kariko K, Ni H, Capodici J, Lamphier M, & Weissman D (2004) mRNA is an endogenous ligand for Toll-like receptor 3. J Biol Chem 279(13):12542-12550.
32. Yanai H, et al. (2009) HMGB proteins function as universal sentinels for nucleic-acid-mediated innate immune responses. Nature 462(7269):99-103.
33. Bsibsi M, et al. (2010) The microtubule regulator stathmin is an endogenous protein agonist for TLR3. J Immunol 184(12):6929-6937.
34. Fabricant C G, Fabricant J, Litrenta M M, & Minick C R (1978) Virus-induced atherosclerosis. J Exp Med 148(1):335-340.
35. Naghavi M, et al. (2003) Influenza infection exerts prominent inflammatory and thrombotic effects on the atherosclerotic plaques of apolipoprotein E-deficient mice. Circulation 107(5):762-768.
36. Krebs P, et al., (2007) Chronic Immune Reactivity Against Persisting Microbial Antigen in the Vasculature Exacerbates Atherosclerotic Lesion Formation. Arteriosclerosis, Thrombosis, and Vascular Biology 27(10):2206-2213.
37. Axtell R C, et al. (2010) T helper type 1 and 17 cells determine efficacy of interferon-beta in multiple sclerosis and experimental encephalomyelitis. Nat Med 16(4):406-412.
38. Zhang L N, et al., (2008) Interferon-beta attenuates angiotensin II-accelerated atherosclerosis and vascular remodeling in apolipoprotein E deficient mice. Atherosclerosis 197(1):204-211.
39. Goossens P, et al. (2010) Myeloid type I interferon signaling promotes atherosclerosis by stimulating macrophage recruitment to lesions. Cell Metab 12(2): 142-153.
40. Bsibsi M, et al. (2006) Toll-like receptor 3 on adult human astrocytes triggers production of neuroprotective mediators. Glia 53(7):688-695.
41. Koga N, et al. (2004) Blockade of the interaction between PD-1 and PD-L1 accelerates graft arterial disease in cardiac allografts. Arterioscler Thromb Vasc Biol 24(11):2057-2062.
42. Groschel S, et al. (2008) TLR-mediated induction of negative regulatory ligands on dendritic cells. J Mol Med 86(4):443-455.

1. (canceled)

2. (canceled)

3. A method for treating or aiding in preventing cardiovascular disease in a patient, comprising the step of administering to the patient a therapeutically effective amount of an agonist of an endosomal TLR.

4. The method of claim 3 wherein the agonist of an endosomal TLR is an agonist of TLR3, optionally poly l:poly Cl₂U.

5. The method of claim 3 wherein the agonist of an endosomal TLR is an agonist of TLR7, TLR8 or TLR9.

6. The method of claim 3 wherein the patient is a patient at risk of restenosis, and/or wherein the patient has or is at risk of atherosclerosis or aneurysm.

7. The method of claim 3 wherein the patient is administered a lipid lowering drug, an oral or injectable antidiabetic treatment and/or a blood pressure lowering drug, and/or an antithrombotic therapy.

8. (canceled)

9. A method for treating or aiding in preventing obesity in a patient, comprising the step of administering to the patient a therapeutically effective amount of an agonist of an endosomal TLR.

10. The method of claim 9 wherein the wherein the agonist of an endosomal TLR is an agonist of TLR3, optionally poly l:poly C12U.

11. The method of claim 9 wherein the patient is administered a lipid lowering drug, an oral or injectable antidiabetic treatment and/or a blood pressure lowering drug.

12. (canceled)

13. A composition or a kit of parts comprising (i) an agonist of an endosomal TLR, and (ii) a lipid lowering drug, an oral or injectable antidiabetic treatment and/or a blood pressure lowering drug and/or an antithrombotic therapy.

14. A method for selecting a compound expected to be useful in treating or aiding in preventing cardiovascular disease or obesity, the method comprising the step of selecting a compound that is an agonist of an endosomal TLR.


SEQUENCE LISTING (3 sequences)

SEQ ID NO: 1 - RNA, 20 nt, Artificial Sequence
  Gu-rich sequence of ssRNA40
gcccgucuguugugugacuc

SEQ ID NO: 2 - DNA, 24 bp, Artificial Sequence
  ODN2006 synthetic oligonucleotide
tcgtcgttttgtcgttttgtcgtt

SEQ ID NO: 3 - DNA, 20 bp, Artificial Sequence
  ODN2216 synthetic oligonucleotide
gggggacgatcgtcgggggg